(19) United States
(12) Patent Application Publication
(10) Pub. No.: US 2009/0238760 A1
(43) Pub. Date: Sep. 24, 2009

(54) ANTISPERMATOGENIC, SPERMICIDAL AND/OR ANTIFUNGAL COMPOSITION AND METHODS OF USING THE SAME

(75) Inventors: Clarence Edgar Cook, Staunton, VA (US); Kevin M Cook, Fairfax Station, VA (US); Clyde Ray Tallent, Raleigh, NC (US); Brian Frazier Thomas, Durham, NC (US); Hernan Andres Navarro, Chapel Hill, NC (US)

Correspondence Address: OBLON, SPIVAK, MCCLELLAND MAIER & NEUSTADT, P.C., 1940 DUKE STREET, ALEXANDRIA, VA, 22314, US

(73) Assignee: Research Triangle Institute, Research Triangle Park, NC (US)

(21) Appl. No.: 12/297,717

(22) PCT Filed: Apr. 19, 2007
(86) PCT No.: PCT/US07/66956

§ 371 Date: Dec. 8, 2008

Related U.S. Application Data
(60) Provisional application No. 60/792,958, filed on Apr. 19, 2006.

(51) Int. Cl. A61K 51/04; C07D 221/16; A61K 31/435; A01N 43/42; G01N 33/567

(52) U.S. Cl. 424/1.85; 546/111; 514/290; 435/7.21

(57) ABSTRACT

Hexahydroindenopyridine compounds are disclosed which act as contraceptive agents by disrupting spermatogenesis, acting as spermicides or sperm motility inhibitors and/or act as antifungals; antispermatogenic, sperm motility inhibitors, spermicidal or antifungal compositions containing the compounds; and methods for disrupting spermatogenesis, inhibiting sperm motility, killing motile sperm or treating fungi using the compounds and compositions. Also disclosed are radioactive compounds which may be used to study the binding of antifertility compounds to specific sites in the body and the use of such compounds for that purpose.

### BACKGROUND OF THE INVENTION

1. Field of Invention

The present invention is directed to hexahydroindenopyridine compounds which act as contraceptive agents by disrupting spermatogenesis, acting as spermicides or sperm motility inhibitors and/or act as antifungals; antispermatogenic, sperm motility inhibitors, spermicidal or antifungal compositions containing the compounds; methods for disrupting spermatogenesis, inhibiting sperm motility, killing motile sperm or treating fungi using the compounds and compositions; and methods and materials for examining the binding of contraceptive agents to biological materials.

2. Discussion of the Background

In the US and much of the Western world, the high and growing demand for contraceptives is a function of lifestyle preferences, while in many developing nations, population control is a highly pressing public health concern. Considering that contraception is a global health need, albeit for different reasons in different parts of the world, the total market for a male contraceptive could be much larger than that accounted for in the US figures alone.

In the Western World, the market for contraceptives has changed relatively little over the past 50 years, with “the pill” having been developed in 1951 and remaining unparalleled as the popular choice for contraception. Advances in contraception research have only offered a few more choices, all to women, who have historically born the brunt of the responsibility, cost, and health-risk (in particular, risks of cardiovascular disease and certain types of cancer associated with long-term use of hormonal-based contraceptives) of contraception. The condom, invented in the 16th century, is the only significant form of contraception available to men (other than “withdrawal” and vasectomy). The only real innovative improvement that has occurred with the condom came with the vulcanization of rubber in the 19th century.¹

As pharmaceutical companies develop compounds for sexual dysfunction (e.g., Viagra for erectile dysfunction), the demand for contraceptives is expected to grow. Condom sales in the U.S. rose 5.8% during 1999, generating $260 million in revenue, reflecting what some have called a new American sexual revolution.² Although the majority of women of child-bearing age already practice contraception³, still half of all pregnancies are unintended.⁴ There has been a persistent appeal from health and consumer groups for more alternatives, in particular for alternatives that allow men to assume a greater portion of contraceptive responsibility. Global concerns have led organizations such as the World Health Organization and Family Health International to launch initiatives aimed at encouraging the development of male contraceptives. At least two companies. Schering and Organon, are investing heavily to bring a hormonal male contraceptive to market within this decade.

Safe and effective orally active male contraceptive drugs have been sought for many years. However, the development of a drug which can safely interrupt spermatogenesis without affecting libido and thereby function as a male contraceptive agent has proven to be a difficult task.

An ideal contraceptive for the male would be one that effectively arrests the production of spermatozoa, blocks their fertilizing capacity without affecting libido or accessory sex organs and their functions, and/or kills motile spermatozoa. In addition it should have a wide separation of effective and toxic doses, and the method should be reversible. Such an ideal male contraceptive agent is currently unavailable.

Some general cellular toxicants such as anticancer agents and alkylating agents affect spermatogenesis, but are obviously not acceptable as contraceptives. Compounds which interfere with cellular energy processes, such as thiosugars that also interfere with spermatogenesis, are not sufficiently selective. Androgens such as testosterone and its analogs, when given in sufficiently high doses, interfere with spermatogenesis, probably through a mechanism involving the hypothalamic-pituitary axis. These steroid compounds have been used successfully in clinical studies. However, the anabolic properties of these steroids may give rise to undesirable side effects. Progestins also presumably act through the hypothalamic-pituitary axis and are effective contraceptive agents in the male, but also require androgen supplementation. ¹ From New Scientist, 20 Apr. 1994, Vol 142 No 1923. ² Drug Store News, Nov. 29, 1999 v21 il9 p 29. ³ 58% of all married women in the world of reproductive age use some of contraceptive method (The Population Division of the United Nations Department of Economic and Social Affairs 2000). ⁴ NICHD, Contraception and Reproductive Health Branch: Report to the NICHHD Council September 1999.

Gonadotrophin releasing hormone (GNRH) analogs have been actively investigated as compounds which effectively block spermatogenesis. However, GNRH analogs interfere with endogenous testosterone production and thus decrease libido unless supplementary androgens are administered.

One approach to male contraceptives is based on identification and exploitation of the biochemistry of the male reproductive process. The testis consists of three functional compartments. The first, responsible for the production of sperm, consists of seminiferous tubules which contain developing germ cells. The second is the Sertoli cell, also located inside the seminiferous tubule, which contributes to the organizational and functional coordination of the spermatogenic process and probably has paracrine and autocrine roles. Due to the complex organizational relationship between the Sertoli cell and the developing germ cells, and the presence of tight junctions between neighboring Sertoli cells, a blood testis barrier is formed, dividing the seminiferous tubule into areas that are isolated from the direct access by blood-borne chemicals or nutrients. Surrounding the tubules, in the interstitial tissue, are Leydig cells that have several endocrine and paracrine functions, the production of testosterone being the best described.

The germinal cells divide and differentiate progressively, moving as they mature from the basement membrane to the tubule lumen. Spermatogonia lie in the basal compartment, and selectively recruited spermatogonia divide mitotically to become either cells that persist as spermatogonia or differentiate into primary spermatocytes. The primary spermatocytes migrate through the junctions between the Sertoli cells and divide meiotically to form secondary spermatocytes. Secondary spermatocytes divide to form spermatids. The spermatids then differentiate into mature spermatozoa. Differentiation of the spermatids is often termed spermatogenesis. However, for the purposes of this application, “spermatogenesis” is defined to cover the entire process of formation and maturation (differentiation) of sperm and an “antispermatogenic compound” is one which disrupts any part of this process.

A summary of Sertoli cell functions is as follows: (a) provide support and nutrition to the seminiferous epithelium, (b) release of late spermatids into the tubule lumen, (c) formation of a morphological and physiologic blood testes barrier, (d) phagocytosis of degenerating germ cells, and (e) regulation of the cycle of seminiferous epithelium.

The Leydig cell also supports spermatogenesis. Luteinizing hormone (LH) from the pituitary stimulates testosterone production by the Leydig cell. Testosterone and its metabolite, dihydrotestosterone, are necessary to support normal spermatogenesis. Testosterone receptors are present on various germ cell types. Testosterone is delivered through the blood testis barrier, likely through transport into the Sertoli cell, where it is metabolized into estradiol, dihydrotestosterone, or remains unaltered.

Some, if not all of the germ cell types, interact with the Leydig and/or Sertoli cell. These interactions are in the form of chemical messengers that are produced by Sertoli, Leydig, and germ cell(s). For example, the pachytene spermatocyte modulates the secretion of a Sertoli cell proteinaeeous factor that in turn stimulates steroidogenesis by the Leydig cell. The binding of spermatids occurs only to Sertoli cells which are rendered competent or functional by exposure to FSH. The Sertoli cell of rats secretes several proteins in a cyclic fashion, with maximal production occurring at a specific stage of the seminiferous epithelium; that is, when it is in association with a specific group of germ cells. Clusterin is produced maximally by Sertoli cells when the seminiferous epithelium is in a Stage VII or VIII configuration that is independent of FSH stimulation, suggesting a local regulation of Sertoli secretory function by germ cells.

Hexahydroindenopyridine compound no. 20-438 developed by Sandoz, Ltd. (see FIG. 1) has been shown to provide reversible inhibition of spermatogenesis on oral administration to animals. See Arch. Toxicol. Suppl, 1984, 7:171-173; Arch. Toxicol. Suppl., 1978, 1:323-326; and Mutation Research, 1979, 66:113-127.

The synthesis of a variety of indenopyridine compounds as racemic mixtures is known and described, for example, in U.S. Pat. Nos. 2,470,108; 2,470,109; 2,546,652; 3,627,773; 3,678,057; 3,462,443; 3,408,353; 3,497,517; 3,574,686; 3,678,058 and 3,991,066. These indenopyridine compounds have a variety of uses including use as serotonin antagonists exhibiting antiphlogistic and analgesic properties, hematoblast aggregation inhibitors, sedatives, and neuroleptic compounds as well as ulcer-protective, hypotensive and anorexigenic compounds.

U.S. Pat. Nos. 5,319,084 and 5,952,336 disclose hexahydroindenopyridine compounds having antispermatogenic activity in which the 5-position is substituted with a phenyl ring having a para-position substituent, such as is shown in structures RTI-4587-056 and RTI-4587-073 in FIG. 1.

Despite extensive research in this field, a need continues to exist for active reversible male antifertility drugs which have limited side-effects. A continuing problem is the need to administer known compounds at dosage levels which may cause side-effects. An additional problem in this field is the lack of suitable imaging agents having specific binding sites on or in the testes. A need continues to exist for compounds which may be used as imaging agents in the study of testicular function and in the diagnosis of testicular malfunction.

In addition to male oral contraceptives, there is a need for more effective spermicidal compositions for use as traditional topical/external contraception practices.

### SUMMARY OF THE INVENTION

Accordingly, one object of the present invention is to provide an orally active male contraceptive drag which does not affect libido, has high potency and activity, and has minimal side effects or toxicity.

A further object of the present invention is to provide an orally active male contraceptive drug which inhibits spermatogenesis and a method of inhibiting spermatogenesis using this drug.

A further object of the present invention is to provide a composition which acts as a spermicide, killing motile sperm, or a sperm motility inhibitor, thus being effective as a contraceptive agent for external application.

Further objects of the present invention are to provide a compound useful for studying the binding of antifertility indenopyridines and other chemicals to a specific site or sites in the body, particularly in the testes; the use of such a compound for identifying, locating and/or quantitating sites that bind antispermatogenic or spermicidal compounds or inhibitors of sperm motility, and also the use of such a compound for assays that can be used to identify structure-activity relationships so as to aid in the development of new antifertility compounds.

It is a further object of the present invention to provide a composition which acts as an antifungal composition.

These and other of the objects of the present invention have been achieved by the discovery of the hexahydroindenopyridine compounds of the present invention and the discovery that these compounds are highly potent, interrupt spermatogenesis and act as a spermicide on motile sperm, and which exhibit effective anti-fungal properties.

The compounds of the present invention solve one or more of the problems noted above. The compounds of the invention exhibit high potency at lower relative dosages than known compound Sandoz 20-438 and reduce the occurrence of side-effects, such as the sedative effects observed with that compound. Further, the compounds of the invention interact with a macromolecular site in the testes. The compounds of the invention which contain a label, such as a radioactive label, overcome the problem of inadequate imaging agents by providing an imaging agent which is useful in the study of testicular function and the diagnosis of testicular malfunction. Such compounds also provide a means for assessing in vitro the relative affinity and thus likely relative in vivo activity of antifertility compounds that bind to a specific receptor. The compounds of the present invention also can act as a spermicidal agent, killing motile sperm in a highly effective and efficient manner, suggesting their use in a variety of spermicidal compositions. The present invention compounds can also act as an anti-fungal.

### BRIEF DESCRIPTION OF THE DRAWINGS

FIG. 1 shows the structures of some previously known antispermatogenic hexahydroindenopyridine compounds and indicates the stereochemistry and numbering system for these compounds.

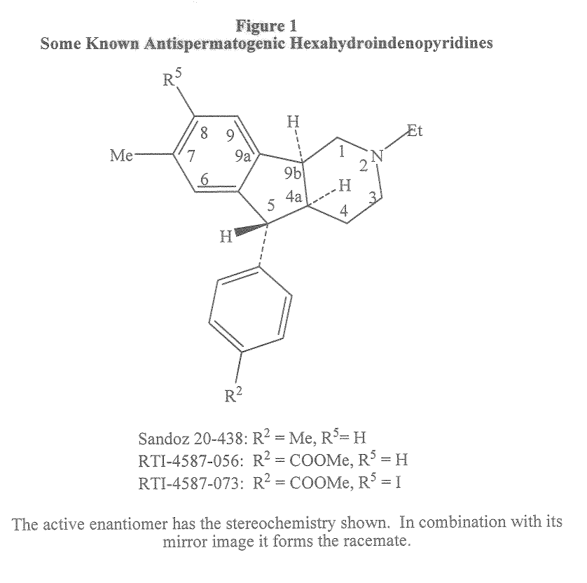

FIG. 2 shows the structures of hexahydroindenopyridine compounds discussed in the biological data and indicates the numbering system for these compounds.

FIG. 3 shows an general outline of processes for preparing compounds of the present invention (Scheme A)

### DETAILED DESCRIPTION OF THE INVENTION

It has now been discovered that hexahydroindenopyridine compounds having the structure 1,

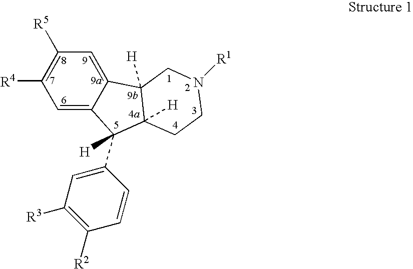

where R¹ is (optionally substituted) C₁₋₄ alkyl, C₃₋₆ cycloalkyl, C₂₋₄ alkynyl, C₂₋₄alkenyl;

R² is a carboxylic ester group in which the alcohol portion of the group has polar substituents or substitution; or

when R⁵ is not Cl, Br, or ¹²⁷I, R² is a carboxylic acid, carboxylic ester or a group (such as for example, methyl, hydroxymethyl, formyl, etc) that can be convened in vivo to a carboxylic acid;

R⁵ is H, halogen, (optionally substituted) C₁₋₄ alkyl, C₃₋₆ cycloalkyl, C₂₋₄ alkynyl, C₂₋₄alkenyl, or OR⁶, where R⁶ is H, C₁₋₄ alkyl, perfluoroalkyl or CₙH_{1 to 2n+1}CO (which may be substituted with fluorine), where n=1 to 8;

R⁴ is C₁₋₄ alkyl, C₃₋₆ cycloalkyl, C₂₋₄ alkynyl, or C₂₋₄ alkenyl, any of which may be substituted with fluorine;

R⁵ is a group containing combinations of C, H, N, O, and S, characterized by one or more π-bonds, which group as it rotates around the C-8/R⁵ bond sweeps out a volume not greater than, that of a cube of dimensions x=y=z=7 Angstroms and which group in addition may be substituted with a group R⁷ that can extend beyond this volume, where R⁷ is a combination of carbon, hydrogen, nitrogen, oxygen and sulfur atoms CₘHₒNₚO_{q}Sᵣ where m=0 to 12, o=0 to 25, p=0 to 5, q=0 to 5, and r==0 to 2; or

R⁵ is R¹¹R¹²R¹³Sn— where R¹¹, R¹², and R¹³ are each, independently, C₁₋₆alkyl, C₂₋₆-alkenyl or C₂₋₆alkynyl; or

R⁵ is ¹²⁵I, ¹²³I, ¹³¹I, ⁷⁶Br, ⁷⁷Br, ¹⁸F, ¹⁹F or ³H; or

when R² is not COOH, COO—CₙH₂ₙ₊₁ (where n is an integer from 1 to 4), CH₂OH, or CHO, R⁵ is Cl, Br, or ¹²⁷I

and salts thereof.

are not only antispermatogenic but also act as a spermicide to kill motile sperm or inhibit sperm motility, and also act as an anti-fungal agent.

Preferably, the compounds of the present invention have structure 1, where R¹ is (optionally substituted) C₁₋₄ alkyl, C₃₋₆ cycloalkyl. 2-4 alkynyl, C₂₋₄ alkenyl;

R² is a carboxylic acid ester wherein the alcohol portion of the ester contains ketal and/or hydroxyl-substituents or a group capable of being converted in vivo to hydroxyl; or

R² (when R⁵ is not Cl, Br, or ¹²⁷I) is a carboxyl or carboxyl ester group COO—CₙH₂ₙ₊₁ (where n is an integer from 0 to 4), CH₂OH, or CHO;

R³ is H, halogen, (optionally substituted) C₁₋₄ alkyl, C₃₋₆ cycloalkyl, C₂₋₄ alkynyl, C₂₋₄alkenyl, or OR⁶, where R⁶ is H, C₁₋₄alkyl, perfluoroalkyl or CₙH_{1 to 2n+1}CO (which may be substituted with fluorine), where n=1 to 8;

R⁴ is C₁₋₄ alkyl, C₃₋₆ cycloalkyl, C₂₋₄ alkynyl, or C₂₋₄ alkenyl, any of which may be substituted with fluorine;

R⁵ is halogen (where R² is not COOH, COOCₙH₂ₙ₊₂ (where n is an integer from 1 to 4), hydroxymethyl or formyl), or R³ is azido or cyano or a group containing C₂₋₄ alkynyl, C₂₋₄alkenyl, single ring aryl or single 5- or 6-membered ring heteroaryl or dihydroheteroaryl, wherein the said group may be substituted with a group R⁷, where R⁷ is a combination of carbon, hydrogen, nitrogen, oxygen and sulfur atoms CₘHₒNₚO_{q}Sᵣ where m=0 to 12, o=0 to 25, p=0 to 5, q=0 to 5, and r=0 to 2; or

R⁵ is R¹¹R¹²R¹³Sn— where R¹¹R¹², and R¹³ are each, independently, C₁₋₆-alkyl, C₂₋₆-alkenyl or C₂₋₆-alkynyl; or

R⁵ is ¹²⁵I, ¹²³I, ¹³¹I, ⁷⁶Br, ⁷⁷Br, ¹⁸F, ¹⁹F or ³H;

and salts thereof.

Still more preferably, the compounds of the present invention have structure 1, where

R¹ is (optionally substituted) C₁₋₄ alkyl, C₃₋₆ cycloalkyl, C₂₋₄ alkynyl, C₂₋₄ alkenyl;

R² is COOR⁸, wherein R⁸ is 2-hydroxypropyl, 3-hydroxypropyl, 2,3-dihydroxypropyl, or CH₂-(2,2-dimethyl-1,3-dioxolan-4-yl), where R⁸ may be R, S or R,S if a chiral center is present;

or if R⁵ is not Cl, Br or ¹²⁷I, R⁸ is H or CₜHᵤ, where t= and integer from 1-18 and u=an integer from 3-37;

R³ is H, halogen, (optionally substituted) C₁₋₄ alkyl, C₃₋₆ cycloalkyl, C₂₋₄ alkynyl, C₂₋₄ alkenyl, or OR⁶, where R⁶ is H, C₁₋₄ alkyl, perfluoroalkyl or CₙH_{1 to 2n+1}CO (which may be substituted with fluorine), where n=1 to 8;

R⁴ is C₁₋₄ alkyl, C₃₋₆ cycloalkyl, C₂₋₄ alkynyl, or C₂₋₄ alkenyl, any of which may be substituted with fluorine;

R³ is a group containing C₂₋₄ alkynyl, C₂₋₄ alkenyl, single ring aryl or single 5- or 6-membered ring heteroaryl or dihydroheteroaryl, wherein the said group may be substituted with a group R⁷, where R⁷ is a combination of carbon, hydrogen, nitrogen, oxygen and sulfur atoms CₘHₒNₚO_{q}Sᵣ where m=0 to 12, o=0 to 25, p=0 to 5, q=0 to 5, and r=0 to 2; or

R⁵ is R¹¹R¹²R¹³Sn— where R¹¹R¹², and R¹³ are each, independently, C₁₋₆-alkyl, C₂₋₆-alkenyl or C₂₋₆-alkynyl; or

R⁵ is ¹²⁵I, ¹²³I, ¹³¹I, ⁷⁶Br, ⁷⁷Br, ¹⁸F, ¹⁹F or ³H; or

when R² is not COOH, COO—CₙH₂ₙ₊₁ (where n is an integer from 1 to 4), CH₂OH, or CHO), R⁵ is F, Cl, Br, or ¹²⁷I

and salts thereof.

In more preferred embodiments, the compounds of the present invention have structure 1, where

R¹ is (optionally substituted) C₁₋₄ alkyl, C₃₋₅ cycloalkyl, C₂₋₄ alkynyl, C₂₋₄ alkenyl;

R² is COOR⁸, wherein R⁸ is 2-hydroxypropyl, 3-hydroxy-propyl, 2,3-dihydroxypropyl, or CH₂₋(2,2-dimethyl-1,3-dioxolan-4-yl), where R⁸ may be R, S or R,S if a chiral center is present; or

when R⁵ is not Cl, Br, or ¹²⁷I, R²═COOR⁹, where R⁹ is H, methyl, ethyl or propyl;

R³ is H, halogen, (optionally substituted) C₁₋₄ alkyl, C₃₋₆ cycloalkyl, C₂₋₄ alkynyl, C₂₋₄ alkenyl, or OR⁶, where R⁶ is H, C₁₋₄ alkyl, perfluoroalkyl or CₙH_{1 to 2n+1}CO (which may be substituted with fluorine), where n=1 to 8;

R⁴ is C₁₋₄ alkyl, C₃₋₆ cycloalkyl, C₂₋₄ alkynyl, C₂₋₄ alkenyl, which may be substituted with fluorine;

R⁵ is azido, cyano, ethynyl or a group containing C₂₋₄ alkynyl, C₂₋₄ alkenyl, single ring aryl or single 5- or 6-membered ring heteroaryl or dihydroheteroaryl, wherein the said group is substituted with a group R⁷, where R⁷ is a combination of carbon, hydrogen, nitrogen, oxygen and sulfur atoms CₘHₒNₚO_{q}Sᵣ where M=1 to 12, o=0 to 25, p=0 to 5, q=0 to 5, and r=0 to 2; or

R⁵ is ¹²⁵I, ¹²³I, ¹³¹I, ⁷⁶Br, ⁷⁷Br, ¹⁸F, ¹⁹F or ³H; or

when R² is not COOH, COO—CₙH₂ₙ₊₁ (where n is an integer from 1 to 4), CH₂OH, or CHO, R⁵ is F, Cl, Br, or ¹²⁷I;

and salts thereof.

In a further embodiment, the compounds of the present invention have structure 1, where

R¹ is methyl, ethyl, n-propyl, i-propyl, allyl or cyclopropyl;

R² is COOR⁸, wherein R⁸ is 2-hydroxypropyl, 3-hydroxypropyl, 2,3-dihydroxypropyl, or CH₂₋(2,2-dimethyl-1,3-dioxolan-4-yl), where R⁸ may be R, S or R,S if a chiral center is present; or

when R⁵ is not Cl, Br, or ¹²⁷I, R²═COOR⁸, where R⁸ is H, methyl ethyl or propyl; R³ is H, halogen, CH₃, CF₃, CHO, COCH₃, OH, OCH₃ or OCF₃;

R⁴ is CH₃, CF₃ or C₂H₅;

R⁵ is halogen, azido, cyano, ethynyl, propynyl, ethenyl, propenyl, triazol-4-yl, C₂₋₄ alkenyl, single ring aryl or single 5- or 6-membered ring heteroaryl or dihydroheteroaryl, wherein the said group where chemically possible may be substituted with a group R⁷, where R⁷ is a combination of carbon, hydrogen, nitrogen, oxygen and sulfur atoms CₘHₒNₚO_{q}Sᵣ where m=o to 12, o=0 to 25, p=0 to 5, q=0 to 5, and r=0 to 2; or

when R² is not COOH, COO—CₙH₂ₙ₊₁ (where n is an integer from 1 to 4), CH₂OH, or CHO, R⁵ is F, Cl, Br, or ¹²⁷I; or

R⁵ is ¹²⁵I, ¹²³I, ¹³¹I,

and salts thereof.

In a further embodiment, the compounds of the present invention have structure 1, where

R¹ is ethyl;

R² is COOR⁸, wherein R⁸ is 2-hydroxypropyl, 3-hydroxy-propyl, 2,3-dihydroxypropyl, or CH₂₋(2,2-Dimethyl-1,3-dioxolan-4-yl), where R⁸ may be R, S or R,S if a chiral center is present;

R³ is H;

R⁴ is methyl;

R⁵ is halogen, trimethylstannyl, ethynyl or (1-(2-(N-piperidino)ethyl))-1H-1,2,3-triazol-4-yl;

and salts thereof.

In a further embodiment, the compounds of the present invention have structure 1, where

R¹ is ethyl;

R² is COOR⁸, wherein R⁸ is H, methyl, ethyl or propyl

R³ is H;

R⁴ is methyl;

R⁵ is trimethylstannyl, ethynyl or (1-(2-(N-piperidino)ethyl))-1H-1,2,3-triazol-4-yl; or

R⁵ is ¹²⁵I, ¹²³I, or ¹³¹I;

and salts thereof.

In a most preferred embodiment of the present invention, the compounds have structure 1, where

R¹ is ethyl;

R² is COOR⁸, wherein R⁸ is 2-hydroxypropyl, 3-hydroxypropyl, 2,3-dihydroxypropyl, or CH₂-(2,2-Dimethyl-1,3-dioxolan-4-yl), where R⁸ may be R, S or R,S if a chiral center is present; or

if R⁵ is not ¹²⁷I, R² is COOR⁸ where R⁸ is H, methyl, ethyl or propyl;

R³ is H;

R⁴ is methyl;

R⁵ is trimethylstannyl, ¹²³I, ¹²⁵I, ¹²⁷I, ¹³¹I, ethynyl or (1-(2-(N-piperidino)ethyl))-1H-1,2,3-triazol-4-yl;

and salts thereof.

The compounds can be used for control of fertility in animals or humans, particularly in mammals, and most preferably in male humans. Alternatively, the compounds can be used for control of fertility in a feral, domesticated or wild animal, preferably in an animal selected from mouse, rat, coyote, dingo, burro, deer, groundhog, coyote or horse.

Within the context of the present invention, the term “spermicide” or “spermicidal”, refers to the ability to either kill spermatozoa or to sufficiently inhibit its motility such that the spermatozoa is unable to fertilize an ovum, and is thus effectively (or functionally) killed.

The present invention further relates to a method for killing or inhibiting the motility of motile spermatozoa, comprising:
contacting a spermatozoa containing composition with a spermicidal composition comprising a compound of structure 1.

In a further embodiment, the invention relates to a spermicidal composition comprising an effective spermicidal dose of a compound of structure 1 and a physiologically acceptable carrier, and the use of such a composition in preparation of a spermicidally treated contraceptive device comprising:

an effective spermicidal amount of a compound of structure 1

and a carrier; and

with or without a contraceptive barrier device.

The composition of the present invention can be used in a contraceptive method, comprising:

orally administering to a subject, a composition comprising an effective spermicidal amount of a first compound of structure 1:

in a pharmacologically acceptable carrier; and

concurrent use by said subject or the sexual partner of said subject of a spermicidal treated contraceptive device comprising:

an effective spermicidal amount of a second compound and a carrier; and

with or without a contraceptive barrier device;

wherein said first compound of formula I(a) and said second compound may be the same or different.

In a further embodiment a compound of the present invention where a substituent is a radioactive atom may be used for studying the mechanism of action of the hexahydroindenopyridine compounds by means of its binding to macromolecular compounds in the body or in vitro, or may be used in assays to test other hexahydroindenopyridines or other compounds for such binding as a means of developing other drugs. For example, a compound of the present invention, wherein R¹, R², R³, R⁴ and R⁵ are as defined above and the compound is rendered radioactive by substitution with a radioactive atom, can be used for identifying, locating or quantitating sites and molecules which bind antispermatogenic or spermicidal compounds. Such compounds can also be used in assays to identify structure-activity relationships among possible antispermatogenic or spermicidal compounds and to aid in the identification and development of new antispermatogenic or spermicidal compounds, as well as in binding assays to determine the relative affinity of compounds for a receptor. More preferably, the compounds of the present invention for use in such methods are those where R¹ is Et, R² is —COOMe or —COOH, R³ is H, R⁴ is Me and R⁵ is ¹²³I, ¹²⁵I, or ¹³¹I, most preferably where R³ is ¹²⁵I. The binding sites or receptors being tested are preferably in the testis or subtractions thereof. Subtractions include, but are not limited to, testicular homogenates, specific cellular components of the testis, or membrane fractions of specific cellular components of the testis.

Present data indicate that a carboxylic ester at the 4′-position of the 5-aryl group in hexahydroindenopyridines is hydrolyzed in vivo to the carboxylic acid, which is believed to be the active moiety in affecting spermatogenesis. Previous work had reported that the use of hydrocarbon moieties (e.g., methyl, n-propyl) as the alcohol component of the ester was effective in antispermatogenesis (Structure-Activity Studies of 2,3,4,4a,5,9b-Hexahydroindeno[1,2-c]pyridines as Antispermatogenic Agents for Male Contraception, C. E. Cook. M. C. Wani, J. M. Jump, Y.-W. Lee, P. A. Fail, S. A. Anderson, Y.-Q. Gu, and V. Petrow. J. Med. Chem., 38(5), 753-763, 1995). We have now discovered that introducing polar groups into the alcohol component also gives effective antispermatogenic agents. Such polar groups may improve solubility properties, aid formulation into drugs and affect the rate of conversion in vivo to the carboxylic acid. It was known that increasing the hydrocarbon chain from methyl to n-propyl resulted in retention of activity. We found that the n-hexyl ester (RTI-4587-101) analogous to RTI-4587-073 also had activity. The more polar esters were all active. These esters were based on the Solketal® moiety and the diol hydrolytic products from it, as well as the 3-hydroxypropyl moiety. Other polar groups may also be used. For example, ester groups containing amine functions, carboxylate functions and their salts would have enhanced polarity.

Within the context of the present invention, the term “anti-spermatogenic” relates to the ability to disrupt the production of sperm in the testes, while the term “spermicide” or “spermicidal” relates to the ability to kill motile sperm or render them immotile after their production and, more preferably, after ejaculation.

Pharmaceutically acceptable salts of the compounds having structure (I) shown above are also included within this invention. Pharmaceutically acceptable salts include, but are not limited to salts of a basic functional group (such as but not limited to an amine group) with inorganic acids such as hydrochloride, hydroiodide, sulphate, phosphate, diphosphate, hydrobromide and nitrate or salts of the basic functional group with an organic acid such as acetate, malate, maleate, fumarate, tartrate, succinate, citrate, lactate, methanesulfonate, p-toluenesulfonate, palmoate, salicylate and stearate; or salts of an acidic functional group with metal ions such as (but not limited to) Na, K, Ca or salts of an acidic functional group with the ammonium ion, or salts of an acidic functional group with organic ions such as (but not limited to) amines and tetrasubstituted ammonium ions.

Hexahydroindenopyridines have three asymmetric centers which can be defined using known nomenclature. Alternatively the relative stereochemistry can be defined by the cis-trans relationships of the hydrogen atoms bonded to the carbon system at positions 4a, 5 and 9b of the tricyclic ring system, leading to stereochemical assignments. The compounds of the present invention have the relative stereochemistry shown in structure (I). This invention includes both individual enantiomeric forms (essentially optically pure) as well as any mixtures of these forms, for example, a racemic mixture.

The antispermatogenic activity of compounds of structure 1 typically resides essentially exclusively in one optical isomer. The measured optical rotation of these compounds, however, may be either (+) or (−), depending on the substitution pattern and the conditions of measurement, as is known to those skilled in stereochemistry. On the other hand, the antifungal properties of the compounds of the present invention are not stereospecific, with both (+) and (−) isomers active, although their relative activities may differ.

The spermicidal effects of the compounds of the present invention have been found in the antispermatogenic isomers and are believed to be present also in the other isomers.

Administration may be by any conventional means or route, including but not limited to, oral, interperitoneal, intraveneous, subcutaneous, intramuscular, inhalation, buccal and skin penetration. These same administration routes are available for spermicidal and/or antifungal treatments, along with topical administration.

The antispermatogenic activity of Sandoz 20-438 is observed after a single oral dose of 30 mg/kg to rats, drastically reducing the weights of the testes within 24 h. Degenerative changes in the seminiferous tubules are observed. Spermatids became pyenotic, occasionally forming multinucleated associations. Sertoli cells appear to be cytologically normal. It appears that Sandoz 20-438 targets spermatids or the Sertoli cell associated with these spermatids because histologic changes are observed in these spermatids first.

The compounds of the invention were tested in mice for their effects on spermatogenesis three days after a single oral dose by the procedure described in Cook et al (J. Med. Chem., 38(5), 753-763, 1995) below. Compounds active in this test have been shown also to be anti-fertility compounds.

Compounds were screened for antispermatogenic activity by dosing male mice on day 1 with a gavage dose of control vehicle, positive control (compound 1) or compound of the invention. At 72 h after dosing, animals were killed and the testes were excised, trimmed of fat, and weighed. One testis was examined histologically and rated for spermatogenic potential using the Spermatogenic Index [J. M. Whitsett, P. F. Noden, J. Cherry and A. D. Lawton, J. Reprod. Fertih, 72, 277 (1984)], which is a semiquantitative estimate of the sperm producing ability of the testes. The index is based on histological appearance of the spermatogenic cells in the seminiferous tubules. A scale of 1 to 6 is used with 5 to 6 being the normal status. A second assessment was based on the weight of the testes.

Table 1 shows pertinent biological results in terms of the effect on testes weight (TW) and spermatogenic index (SI) compared to a control containing only the administration vehicle, but no indenopyridine.

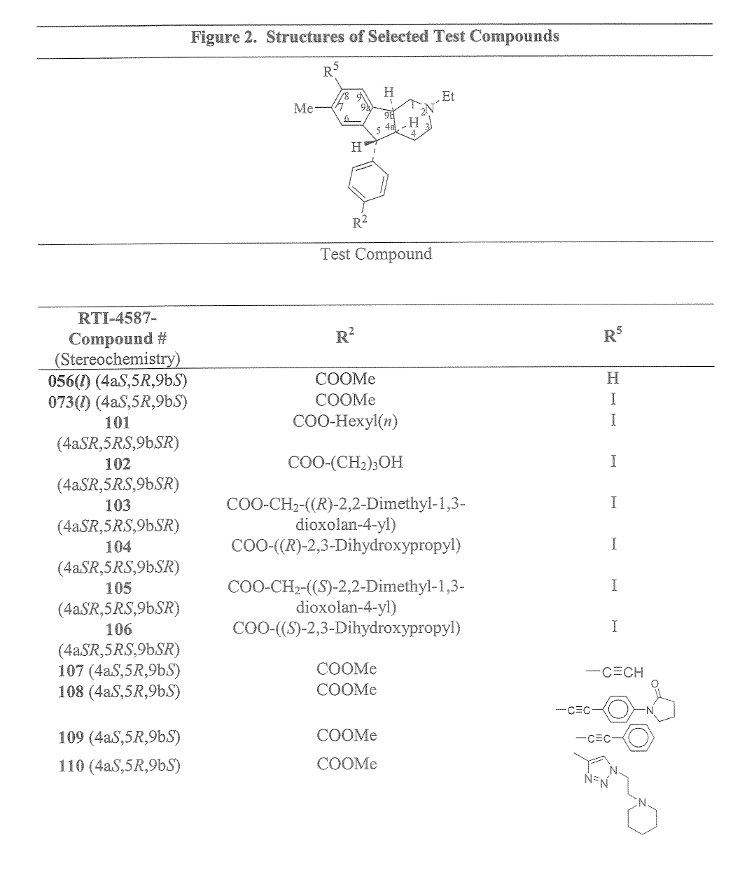

FIG. 2 shows the structures of the compounds of Table 1.

TABLE 1
Testicular Response to Selected Indenopyridine Analogs
in Swiss (CD-1) Mice^{a}

| Test | | | |
|---|---|---|---|
| Compound | Dose^{c} | | Spermatogenic |
| (RTI #4587-)^{b} | (mg/kg) | Testes Weight (mg) | Index |
| Control^{d} | 0 | 204 ± 11.9 | 5.5 ± 0.16 |
| 073(l) | 1 | 144 ± 4.9* | 2.92 ± 0.15* |
| 101 | 1.2 | 154.4 ± 6.2* | 3.93 ± 0.03* |
| 102 | 5 | 161.1 ± 4.1* | 3.06 ± 0.10* |
| 103 | 5 | 156.0 ± 3.2* | 2.92 ± 0.12* |
| 104 | 5 | 170.1 ± 11.7 | 3.04 ± 0.06* |
| 105 | 5 | 152.0 ± 5.9* | 3.91 ± 0.14* |
| 106 | 5 | 168.2 ± 16.4 | 3.20 ± 0.15* |
| 056(l)# | 30 | 173 ± 8.8 | 3.69 ± 0.07* |
| 107 (from 073(l)) | 20 | 134.4 ± 7.0* | 1.56 ± 0.20* |
| 108 (from 073(l)) | 20 | 195.5 ± 12.6 | 5.20 ± 0.06 |
| 109 (from 073(l)) | 20 | 168.1 ± 16.4 | 4.26 ± 0.12* |
| 110 (from 073(l)) | 20 | 133.0 ± 5.7* | 2.75 ± 0.05* |

^{a}Numbers are mean ± standard error of the mean. The number of observations equals 5 unless otherwise stated. A single dose was given orally and the animals killed 72 h later.
^{b}See FIG. 2 for structures and stereochemistry.
^{c}Racemic compounds presumably have only ½ the dose shown of active ingredient.
^{d}Control data are for vehicle-dosed animals.
*Significantly different from control (Dunnett's one-tailed t-test, p < 05).
^{#}n = 4

The spermicidal activity of the compounds of the present invention is preferably present with only a single application of composition to ejaculate. The spermicidal composition of the present invention can have any concentration sufficient to decrease motility of the spermatozoa to a level insufficient to cause impregnation, preferably a concentration of from 1-500 μM more preferably from 3-300 μM, most preferably from 10-200 μM. Spermicidal activity was determined by the method described below.

Method for Determining Direct Effect of an Agent on Sperm Motility

The direct effect of an agent on sperm motility is determined by using the following protocols. Basically, sperm are obtained either from the Cauda epididymis, as in the case of the rat, or ejaculated sperm collected using an artificial vagina, as in the rabbit. The initial motility of the sperm is determined manually or by using a Hamilton Thorn IVOS sperm analyzer. The sperm are then kept at a constant temperature of 34° C., diluted to a consistent concentration of 10×106/ml, and added to about 3 ml of buffer or media. The motility is determined again at this point and any change is recorded. Different concentrations of the agent to be tested are then added to the sperm preparation. The sperm samples are then held at the same temperature for one hour and the motility is determined. The results are recorded as the percent of motile sperm in the sample.

Protocol for Determination of a Direct Effect of an Agent on Sperm Motility

General:

The Sperm are kept at 34° C. throughout the experimental period. Sperm concentration is approximately 10×10⁶/ml (note, the sample may have to be diluted with buffer or media to reach this concentration).

For the Rat Studies: (Using Sperm from the Cauda Epididymis)

A 1 mM stock of the indenopyridine in HBSS buffer+BSA (5 mg in 10=0.5 ug/ul; 0.5 ug/ml=1 uM) is prepared and added as follows:

1 uM=1 ul stock+949 ul HBSS buffer+BSA+50 ul of the diluted sperm

3 uM=3 ul stock+947 ul HBSS buffer+BSA+50 ul of the diluted sperm

10 uM=10 ul stock+940 ul HBSS buffer+BSA+50 ul of the diluted sperm

30 uM=30 ul stock+920 ul HBSS buffer+BSA+50 ul of the diluted sperm

100 uM=100 ul stock+850 ul HBSS buffer+BSA+50 ul of the diluted sperm

300 uM=300 ul stock+650 ul HBSS buffer+BSA+50 ul of the diluted sperm

1000 uM=1000 ul stock+50 ul of the diluted sperm

Sperm Motility is determined after 1 hour.

### For the Rabbit Studies: (Using Ejaculated Sperm)

A 1 mM stock of the indenopyridine in M-199 media with BSA (2.5 mg in 5=0.5 ug/ul; 0.5 ug/ml=1 uM) is prepared and added as follows:

1 uM=1 ul stock+949 ul M-199 media with BSA+50 ul of the diluted sperm

3 uM=3 ul stock+947 ul M-199 media with BSA+50 ul of the diluted sperm

10 uM=10 ul stock+940 ul M-199 media with BSA+50 ul of the diluted sperm

30 uM=30 ul stock+920 ul M-199 media with BSA+50 ul of the diluted sperm

100 uM=100 ul stock+850 ul M-199 media with BSA+50 ul of the diluted sperm

300 uM=300 ul stock+650 ul M-199 media with BSA+50 ul of the diluted sperm

1000 uM=1000 ul stock+50 ul of the diluted sperm

Sperm motility is determined after 1 hour.

Slides are made to determine morphology changes and eosin is added to determine cell death,

The compounds of the present invention are useful as male antifertility drugs for controlling fertility in mammals, including humans. In addition to their potential use in family planning, the compounds of the invention are also useful to control fertility in domestic, wild or feral animals, where lethal measures are not practical or desirable. For example, the control of deer populations is a problem in some areas of the United States. Oral administration of the compounds of the present invention to seasonal breeding animals such as deer by means of baited feed containing these compounds at appropriate times would substantially reduce reproductive capacity. Other target animals include rodents such as mice or rats, prairie dogs, coyotes, wolves, etc., as well as feral goats, swine, horses, etc. Administration of the compounds of this invention to captive zoo animals provides a means of controlling reproduction in species which become overpopulated.

By “controlling fertility” as used herein is meant reducing the reproductive capacity or fertility of the animal or human treated. The compounds of the invention are administered in a single dose or a plurality (two or more) of doses where the doses are sufficient to reduce the sperm producing ability of the animal or human (spermatogenic index) to a level of infertility. That is, the compounds of the invention are administered in an amount and for a length of time sufficient to reduce the sperm count to a level which is not sufficient to reproduce. The length of infertility is a function of dose such that with sufficient doses one may extend the period of infertility so as to essentially use the compounds of this invention to perform sterilization; thus, the compounds of the invention may replace surgical vasectomy as a means of male sterilization.

For the above-mentioned uses, the dose of the compound of the invention will naturally vary depending on the specific compound employed, the mode of administration and the length of infertility desired. However, satisfactory results are obtained in animals at oral doses from about 0.01 to about 100 mg/kg, preferably about 0.1-25 mg/kg body weight per day. For larger animals, a daily dose amount of about 10-500 mg may be administered as a single oral unit dose or in divided dosage units containing about 5-250 mg of the compound of the present invention. When administering a single active enantiomer, one may generally administer a smaller dose then when administering a racemic compound. If desired or necessary, the compounds of the invention may be administered together with solid or liquid carriers or diluents or in slow-release form. Formulation of these pharmaceuticals forms is well known in the art and any conventional method of preparing solid, liquid and slow-release formulations may be used with the compounds of the present invention. The compounds of the invention may also be administered by means of conventional implants or skin patches which are well known in the art.

The compounds of the invention may be used in human contraception in males, either by reversibly blocking spermatogenesis or in nonsurgical sterilization. In the latter use, administration of appropriately large doses realizes the effects of vasectomy without the use of surgery and with the elimination of potential side effects of vasectomy.

The compounds of the invention are also useful in the control of reproduction in domestic, wild, feral or zoo animals for example, the compounds may be in the control of reproduction in zoo animals. Wild and feral animal populations close to human habitation, for example deer or coyotes, or animal populations which strongly impact the natural ecology, for example wild mustangs and feral hogs, may be controlled by selectively baiting without using lethal means such as shooting or poisoning. Animal behavior is not affected in this process, only fertility.

When R⁵ is a radioactive label, the compounds of the invention are useful to study testicular function and diagnose testicular malfunction. Administration of the compounds in dosages of 0.1 to 10,000 μCi, more preferably 1 to 100 μCi, results in binding to testicular tissue, which can be detected by various means of detecting radiation, as is known in the art.

In their anti-spermatogenic properties, the high degree of chemo-, stereo- and enantioselectivity of the compounds together with their lack of general effects, such as on libido, indicates that they are interacting with a specific macromolecule in the testis. Treatment of testis or testis fractions with a radioactive derivative of the compounds followed by detection of radioactivity by techniques well known in the art of radiochemistry enable one to locate and identify the portion of the testis and the macromolecule involved in the antispermatogenic effect. This may be used to detect and Identify an important constituent of the testis, disruption of which can lead to an antifertility effect. Comparison of the ability of other compounds (such as analogs of the current compounds or those from combinatorial libraries) to inhibit the binding of the radiolabeled compound can lead to even more selective and potent antispermatogenic compounds. Furthermore, by administering a small dose (too small to have a clinical effect on fertility) of the radiolabeled compound to an animal or human subject and then measuring the amount of radioactivity in the testis or specific areas of the testis, one can show whether an existing problem of infertility is related to the lack of this macromolecule. The radioactivity can be measured in a living animal or human by techniques such as PET and SPECT which are well known in the art of imaging of biological tissues.

The compounds are also useful as internal standards for analytical purposes, as described in U.S. application Ser. No. 10/350,232, the contents of which are hereby incorporated by reference.

For use as a spermicide, the compounds of the present invention can be prepared in a variety of forms for administration. Conventional spermicidal composition forms are readily prepared using known methods. Such spermicidal compositions can take the form of gels, foams, jellys, creams, ointments, salves, etc. Conventional carriers are used to prepare the compositions. The present spermicidal compositions can be administered alone or in combination with one or more barrier methods of contraception, such as a diaphragm, sponge or condom. The composition can be applied directly to the diaphragm, sponge or condom immediately prior to use, or can be prepackaged along with the sponge or condom (or even diaphragm, although most diaphragms are for multiple uses and are cleaned between uses).

For use as a fungicide, the composition of the present invention can be prepared in any suitable form for administration to the area in need thereof. The forms of administration include, but are not limited to, those listed above for spermicidal compositions, as well as liquid mixtures. It is also possible for the fungicidal and spermicidal properties to be used in combination using one of the common forms of administration.

Precursors for the compounds of the invention can be prepared by the methods disclosed in U.S. Pat. Nos. 5,319,084 and 5,952,336, and by using modifications of the method disclosed in U.S. Pat. No. 3,678,057. These patents are incorporated herein by reference in their entirety. The mixtures of enantiomers produced by this process are resolved into pure enantiomers by salt formation followed by selective crystallization or chromatography, as described in C. E. Cook et al, J. Med. Chem., 38:753 (1995). Alternatively, an adaptation of the process described in U.S. Pat. No. 5,952,336 may be used. Optical purity is established by high pressure liquid chromatography (HPLC) on a CHIRACEL-OD column.

Compounds of this invention may be prepared as shown in the general Scheme A (FIG. 2) and more specifically by means of the Examples, beginning with structure A3 where R² is COOH or one of its esters and where R⁵ is a halogen such as iodine. Such compounds as A3 are prepared as described in U.S. Pat. Nos. 5,319,084 and 5,952,336. A carboxylic acid may be converted to carbonyl chloride by use of thionyl chloride or other reactions known in the art. Reaction of the carbonyl chloride with an alcohol in the presence of a catalyst such as 4-N,N-dimethylaminopyridine leads to the esters A4. The esters A4 may be further modified by reactions such as deprotection of hydroxyl protecting groups and other reactions known in the art.

If R⁵ of A3 is a halogen, most effectively iodine, and R³ is an ester, preferably methyl ester, compounds of structure A1 may be obtained by reaction with ethynylzinc bromide in the presence of Pd catalysts such as tetrakis(triphenylphosphine)palladium(0) (method of Negishi et al., J. Org. Chem. 62:8597 (1997))). The ethynyl group may be modified by numerous procedures known in the art. Thus, ethynyl groups are known to react by 1,3-cycloaddition reactions to give a variety of heterocyclic compounds. For example, reaction with organic azido compounds leads to triazoles (cf. Rostovtsev, V. V.; Green. L. G.; Fokin, V. V.; Sharpless, B. K. Angew. Chem. Int. Ed., 2002, 41 (14), 2596-2599). Reaction of terminal ethynes with aryl or heteroaryl halides or triflates in the presence of Pd and Cu catalysts (Sonogashira reaction) leads to arylethynes, for example compound A5 where is an (optionally substituted) aryl group. Alkylation of the ethyne may also be accomplished by procedures known to the art. If R⁵ of A3 is a halogen, then reaction with arylboronic acids in the presence of noble metal catalysts (Suzuki reaction) leads to 8-aryl compounds of type A7.

The synthesis of metallated analogs is exemplified by the reaction of 8-iodo compound with hexamethylditin in the presence of tetrakis(triphenylphosphine)palladium(0) to yield compounds of structure A6. Compounds of this type are of special interest for the synthesis of radioiodinated materials for use in binding studies, diagnostics and metabolism and pharmacokinetic studies. Simple reaction with radioactive iodide in the presence of chloramine-T rapidly leads to labeled compounds with high specific activity. One may also obtain other radioactive analogs of the various subject compounds. Tritium-labeled compounds of the invention may be obtained, for example, by reduction of the 8-iodo compounds with tritium gas catalyzed by a noble metal, such as palladium or platinum. Carbon-14 analogs may be made, for example, by using ¹⁴C labeled intermediates in the synthesis. Other methods for isotopic labeling of the compounds commonly used in the art of radiochemical synthesis may also be applied.

The use of compounds substituted with a radioactive atom for locating and identifying specific binding sites (“receptors”) for the compounds has been exemplified in a number of cases. In general the radioactive compound (radioligand) is incubated with a homogenate or subfraction of the homogenate (e.g., membranes, cytosols, or nuclei) from a tissue suspected of containing the receptor, usually at 0 to 37° C. for a period ranging from a few minutes to 24 hours. Then the mixture is treated so as to separate the receptor bound radioactivity from the unbound radioligand. This may include precipitation, filtration, adsorption of the free radioligand or the receptor with bound radioligand and other procedures known to the field of receptor study. Receptor-bound radioactivity is then measured by standard procedures such as in a gamma counter or by use of liquid scintillation spectrometry. Binding not specific to the receptor is determined by adding a large excess of the unlabeled ligand and performing the same procedure. Subtraction of the non-specific radioactivity bound from the total binding shows the specific binding of the radioligand to the receptor. Measuring displacement of the radioligand by unlabeled ligand permits determination of the Kd for the ligand. Comparing the displacement of the radioligand by its unlabeled isotopolog with the displacement of the radioligand by other active compounds makes it possible to compare binding and in vivo potency, and if these are reasonably in agreement this further supports the identification of a receptor. Determining the binding of new compounds then leads to identification of compounds that can have similar activity and may thus be candidates for drug development.

The use of radiolabeled compounds to study the in vivo distribution and binding of radioligands and the changes on disease progression is exemplified by the use of 1-123 altropane (a cocaine analog) for study and potential diagnostic use in patients with Parkinson's disease, as for example in the paper of Fischman et al. [Fischman A J, Bonab A A, Babich J W, Palmer E P, Alpert N M, Elmaleh D R, Callahan R J, Barrow S A, Graham W, Meltzer P C, Hanson R N, and Madras B K (1998). Rapid detection of Parkinson's disease by SPECT with altropane: a selective ligand for dopamine transporters. Synapse 29:128-41.

Other features of the present invention will become apparent in the course of the following descriptions of exemplary embodiments which are given for illustration of the invention and are not intended to be limiting thereof.

### EXAMPLES

Designation of specific manufacturers, instruments, chromatographic material, chemical suppliers, etc. in the examples is for illustration only and does not preclude the use of other, similar items.

Spermatogenic Index Test of Selected Hexahydroindenopyridine Compounds

Compounds were given orally to 8-week old male CD-1 mice in a single dose. Seventy-two hours later the animals were killed and the testes weighed. One testis was subjected to histological analysis and scored according to the Spermatogenic Index (SI) of Fail (see Cook et ah, J. Med. Chem. 38(5), 753-763, 1995 for methodology). All of the new esters were made from the racemic 8-iodo compound RTI-4587-073, and thus have the racemic 4aSR,5RS,9bSR stereochemistry. Compounds RTI-4587-107 through -110 were made from the enantiomeric compound RTI-4587-073(l) and thus have the 4aS,5R,9bS stereochemistry. For comparison one standard group was given the 8-iodo 4′-methoxycarbonyl analog, RTI-4587-073(l), a second standard group was given the 4′-methoxycarbonyl analog. RTI-4587-056(l), and a vehicle control group was also analyzed. Data are given in the Table 1 below:

TABLE 1
Testicular Response to Selected Indenopyridine Analogs
in Swiss (CD-1) Mice^{a}

| Test | | | |
|---|---|---|---|
| Compound | Dose^{c} | | Spermatogenic |
| (RTI #4587-)^{b} | (mg/kg) | Testes Weight (mg) | Index |
| Control^{d} | 0 | 204 ± 11.9 | 5.5 ± 0.16 |
| 073(l) | 1 | 144 ± 4.9* | 2.92 ± 0.15* |
| 101 | 1.2 | 154.4 ± 6.2* | 3.93 ± 0.03* |
| 102 | 5 | 161.1 ± 4.1* | 3.06 ± 0.10* |
| 103 | 5 | 156.0 ± 3.2* | 2.92 ± 0.12* |
| 104 | 5 | 170.1 ± 11.7 | 3.04 ± 0.06* |
| 105 | 5 | 152.0 ± 5.9* | 3.91 ± 0.14* |
| 106 | 5 | 168.2 ± 16.4 | 3.20 ± 0.15* |
| 056(l)# | 30 | 173 ± 8.8 | 3.69 ± 0.07* |
| 107 (from 073(l)) | 20 | 134.4 ± 7.0* | 1.56 ± 0.20* |
| 108 (from 073(l)) | 20 | 195.5 ± 12.6 | 5.20 ± 0.06 |
| 109 (from 073(l)) | 20 | 168.1 ± 16.4 | 4.26 ± 0.12* |
| 110 (from 073(l)) | 20 | 133.0 ± 5.7* | 2.75 ± 0.05* |

^{a}Numbers are mean ± standard error of the mean. The number of observations equals 5 unless otherwise stated. A single dose was given orally and the animals killed 72 h later.
^{b}See FIG. 2 for structures and stereochemistry.
^{c}Racemic compounds presumably have only ½ the dose shown of active ingredient.
^{d}Control data are for vehicle-dosed animals.
*Significantly different from control (Dunnett's one-tailed t-test, p < 05).
^{#}n = 4

Binding and displacement of ¹²⁵I-(4aS, 5R, 9bS)-5-(4-Carbomethoxyphenyl)-2-ethyl-2,3,4,4a,5,9b-hexahydro-8-iodo-7-methyl-1H-indeno[1,2-c]pyridine (¹²⁵I-RTI-4587-074(d) to testis membranes

### 1.0 Testicular Membrane Preparation

### 1.1 Tissue Buffer

### 10 mM TRIS-HCL, pH 7.2

### 1 mM MgCl₂

### 1 mM CaCl₂

(Note: All chemicals were obtained from Sigma Chemical Co., St. Louis, Mo. and are reagent or tissue culture grade. Buffers and tissue should be kept chilled on ice throughout the procedure.)

### 1.2 Tissue Preparation

1. Thaw frozen rat testes (from Sprague-Dawley male rats, 7-8 weeks old, Pel-Freez Biologicals, Rogers, A R) and remove tunica if it was not removed during excision.
2. Weigh the tissue, place in a beaker, and add 5 times the tissue weight in mL of cold tissue buffer above.
3. Homogenize the tissue for 20 sec with a Polytron tissue homogenizer (probe no. PTA 10S, 12 mm) at a medium setting (4-6 on a rheostat scale of 10) or until a smooth suspension is obtained.
4. Pour the homogenate into a chilled 50 ml, centrifuge tube, and centrifuge at 0-4° C. and 500×g (˜1540 rpm in an IEC CENTRA-7R) for 10 min.
5. Decant the supernatant into clean 10 ml, centrifuge tubes, using multiple tubes as necessary.
6. Centrifuge the decanted supernatant at 0-4° C. and 17000×g (˜11900 rpm in a Sorvall SS-34 rotor) for 12 min.
7. Decant and discard the supernatants. Resuspend and combine the pellets in 1.5 times (in mL) the original wet tissue weight in the buffer above. Perform a total protein assay on resuspended pellet.

### 2.0 Biorad Total Protein Assay

Prepare protein standards from bovine serum albumin (BSA, catalog #A-3294 or equivalent, Sigma Chemical Co., St. Louis, Mo.) as follows:

A. 20 mg BSA+10 mL Buffer 3=2.0 mg/mL

B. 0.75 mL A+0.25 mL Buffer 3=1.5 mg/mL

C. 0.50 mL A+0.50 mL Buffer 3=1.0 mg/mL

D. 0.25 mL A+0.25 mL Buffer 3=0.5 mg/mL

E. 0.05 mL A+0.95 mL Buffer 3=0.1 mg/mL

(Store standards at −20° C.)
1. Prepare the Bio-Rad Protein Assay dye reagent (catalog no. 500-0006, Bio-Rad Laboratories, Hercules. CA) by mixing 1 part dye with 4 parts DI/DS H₂O. Filter.
2. Add 40 μL of each standard to a labeled 13×100 mm glass test tube. Prepare a reagent blank by adding 40 uL tissue buffer to a test tube.
3. Add 5-20 mL of tissue preparation to the appropriate test tube. Assay in duplicate.
4. Add 2 mL of dye reagent to each tube and vortex.
5. Allow the reaction mixture to incubate at room temperature between 5 min and 60 min.
6. Revortex, and zero the spectrophotometer with the reagent blank at 595 nM. Measure and record the absorbance of the standards and test samples.
7. Construct a regression curve from the standard absorbances, and determine the concentration of the tissue from the standard curve. Multiply the tissue result by the appropriate dilution factor based on the proportion of 40 uL used.
8. Aliquot the testes tissue preparation into cryovials so that there is sufficient tissue to run one assay set. Store tissue at −70° C.

### 3.0 Scatehard Receptor Binding Assay

The Scatehard assay measures the binding of a radiolabeled test compound, over a wide concentration range, to the membrane receptors in both the absence (total binding) and presence (non-specific binding) of non-radioactive compound. Calculation of the resulting assay data yields the K_{d} of the test compound.

### 3.1 Reagents

| | |
|---|---|
| Assay Buffer: | 10 mM TRIS-HCL, pH 7.2 |
| | 1 mM MgCl₂ |
| | 1 mM CaCl₂ |
| | 5 mg/mL BSA |
| Wash Buffer: | 50 mM TRIS-HCL, pH 7.2 |

### 3.2 Assay Procedure

1. Label 1.2 mL polypropylene tubes in duplicate for each [¹²⁵I]074(d) standard total binding and non-specific binding (NSB) determination and place in 96-well matrix test tube racks. Label liquid scintillation vials for free radioactivity determination.
2. Prepare dilutions of the [¹²⁵I]074(d) ranging in concentration from approximately 1 to 200 nM or other desired concentration range. Prepare cold drug at a concentration of at least 100 times the level of the highest level of radiolabeled standard (e.g. 20-40 □M) for NSB determination.
3. Set up the assay as follows:

| | | | Non-specific |
|---|---|---|---|
| | Free Drug | Total Binding | Binding |
| Component | (mL) | (mL) | (mL) |
| [¹²⁵I]074(d) | 0.05^{a} | 0.05 | 0.05 |
| Dilution | | | |
| Unlabeled 074(d) | — | — | 0.05 |
| Assay Buffer | — | 0.05 | — |

^{a}Add directly to LSC vial, add Ultima Gold scintillation cocktail or equivalent and count.

4. Dilute testes preparation in assay buffer to a concentration of 2 mg/mL of protein. Add 0.1 mL (200 μg) to each assay tube. Vortex to mix.
5. Incubate assay at 25° C. for 2 hours while gently shaking at 30-35 rpm (e.g. Precision Scientific Reciprocal Shaker or equivalent).
6. Place a GF/C 96-well filter plate (glass fiber filter plate catalog no. 6005174, Perk in Elmer LAS, Shelton, Conn.) in assay buffer and allow to soak for at least 30 min.
7. Near the end of the incubation period, place a blank wash plate in the cell harvester (model MPXR-96T, Brandel Scientific, Gaithersburg, Md.). Place cold wash buffer in the reservoir supplying the harvester. Prime the harvester with buffer. Replace the dummy filter plate with the pre-soaked plate.
8. At the end of the incubation period, remove tubes from incubator. Place the harvester prongs into the tubes, and vacuum to deposit the reaction mixture onto the filter plate. Rinse the reaction tubes 3-4 times with cold buffer and vacuum through the filter plate.
9. Remove the filter plate from the harvester and allow it to dry thoroughly, using a warm oven if desired.
10. Seal the bottom of the plate. Insert the plate into the cocktail dispenser and add cocktail (Micro-Scint 20, Perkin Elmer LAS, Shelton, Conn.) to each well
11. Seal the top of the plate. Allow the plate to sit at least one hour or overnight and then count.

### 3.3 Calculations

1. For each level of standard, determine the CP Ms of free (essentially equivalent to total radioactivity added), total bound, and non-specific bound (NSB) compound.
2. Subtract the NSB CPMs from the corresponding total CP Ms for each standard level to determine specific bound for each standard level.
3. Convert the free, specific bound and non-specific bound CPMs to a mass basis (e.g. pM) using the specific activity, counter efficiency for the isotope (˜70% for ¹²⁵I in a Packard Model 1900TR. Liquid Scintillation Analyzer) and assay dilution factor of the labeled material.
4. For each standard level, divide the level of compound specifically bound by the corresponding free amount added.
5. For Scatehard analysis, perform a linear regression analysis where x=specific bound compound and y=ratio of corresponding specific bound/free compound added.
6. The Kd=−1/slope of the line generated.
7. For illustrative purposes, a binding isotherm graph can be generated where x=free compound and y=total bound, non-specific bound and specific bound compound.

### 4.0 Displacement Assay Procedure

The displacement assay measures the ability of a test drug, over a wide concentration range, to compete with and displace a constant amount of a known radiolabeled drug bound to the testes receptor. Calculation of the resulting assay data yields the Kᵢ of the test drug.

### 4.1 Reagents

| | |
|---|---|
| Assay Buffer: | 10 mM TRIS-HCL, pH 7.2 |
| | 1 mM MgCl₂ |
| | 1 mM CaCl₂ |
| | 5 mg/mL BSA |
| Wash Buffer: | 50 mM TRIS-HCL, pH 7.2 |

### 4.2 Assay Procedure

1. Label 1.2 mL polypropylene tubes in duplicate for each drag standard concentration, total binding, and non-specific binding (NSB) determination and place in 96-well matrix test tube racks.
2. Prepare dilutions of the test displacing drug ranging in concentration from approximately 10 nM to 200 mM or other suitable concentration range.
3. Prepare [¹²⁵I]074(d) at a concentration of approximately 20 nM. Prepare 40 uM unlabeled 074(d) for non-specific binding determination.
4. Set up the assay as follows:

| | | | Non-specific |
|---|---|---|---|
| | Displacement | Total Binding | Binding |
| Component | Curve (mL) | (mL) | (mL) |
| [¹²⁵I]074(d) (5 nM) | 0.05^{a} | 0.05 | 0.05 |
| Test drug dilution | 0.05 | — | — |
| (~10 nM-200 nM) | | | |
| Unlabeled 074(d) ( | — | — | 0.05 |
| Assay Buffer | | 0.05 | |

5. Dilute testes preparation in assay buffer to a concentration of 2 mg/mL of protein. Add 0.1 mL (200 μg) to each assay tube. Vortex to mix.
6. Incubate assay at 25° C. for 2 hours while gently shaking at 30-35 rpm (e.g. Precision Scientific Reciprocal Shaker or equivalent).
7. Place a 96-well GF/C filter plate in assay buffer and allow to soak for at least 30 min.
8. Near the end of the incubation period, place a blank wash plate in the cell harvester. Place cold wash buffer in the reservoir supplying the harvester. Prime the harvester with buffer. Replace the dummy filter plate with the pre-soaked plate.
9. At the end of the incubation period, remove tubes from incubator. Place the harvester prongs into the tubes and vacuum the reaction to deposit the mixture onto the filter plate. Rinse the reaction tubes 3-4 times with cold buffer.
10. Remove the filter plate from the harvester and allow it to dry thoroughly, using a warm oven (˜55° C.) if desired.
11. Seal the bottom of the plate. Insert the plate into the cocktail dispenser and add an appropriate amount of cocktail to each well.
12. Seal the top of the plate. Allow the plate to sit several hours or overnight and then count.

### 4.3 Calculations

1. For each level of standard, calculate the CPMs specifically bound from the following equation;

$$\frac{Test drug C P M - N S B C P M}{Total Bound C P M - N S B C P M} \times 100$$

2. Plot the % bound vs. standard concentration using GraphPad Prism™ software or other suitable graphing program. From the non-linear regression, one-site competition fit equation, report the Kb of each test compound.

### 5.0 Results

An example of the results of the binding assay in terms of average Ki for several hexahydroindcnopyridine compounds is given in Table 2. It will be seen that the most potent compound to date. RTI-4587-074(d), has a Ki (Kd in this case) of ca 0.027 micro molar (μM), whereas its analog lacking an 8-iodo substituent, RTI-4587-054(d), has a Ki of ca. 0.54 uM. This order of binding affinity is consistent with the much greater in vivo activity of -074(d), as described in Cook, et ah, J. Med. Chem., 1997, 40, 2111-2112. Also, the enantiomer of -054(d), RTI-4587-054(l), which is inactive in mice in vivo, has a much higher Ki (ca. 330 uM). This is also the case with -002(d) (bioactive, Ki of 6.1 uM) versus -002(l) (bioinactive, Ki of 66 uM). The Ki of -002(d) is much higher than would be expected on the basis of its in vivo activity, which supports the hypothesis that it undergoes metabolism to the effective carboxylic acid species. The carboxylic esters have moderate binding compared with their carboxylic acid analogs, which is again consistent with their metabolism in vivo to the acids.

TABLE 2
Structures of Test Compounds and Binding Affinities

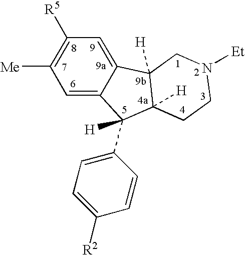

Test Compound

| | | | |
|---|---|---|---|
| RTI-4587- | | | |
| Compound # | | | |
| (Stereochemistry) | R² | R⁵ | Ki (μM) |

| | | | |
|---|---|---|---|
| 002(d) | CH₃ | H | 6.1 |
| (4aS,5R,9bS) | | | |
| 002(l) | CH₃ | H | 65.6 |
| (4aR,5S,9bR) | | | |
| 054(d) | COOH | H | 0.54 |
| (4aS,5R,9bS) | | | |
| 054(l) | COOH | H | 329 |
| (4aR,5S,9bR) | | | |
| 056(d) | COOMe | H | 207 |
| (4aR,5S,9bR) | | | |
| 056(l) | COOMe | H | 2.7 |
| (4aS,5R,9bS) | | | |
| 073(d) | COOMe | I | 67.3 |
| (4aR,5S,9bR) | | | |
| 073(l) | COOMe | I | 0.32 |
| (4aS,5R,9bS) | | | |
| 074(d) | COOH | I | 0.027 |
| (4aS,5R,9bS) | | | |

### Synthetic Examples

General Experimental Conditions

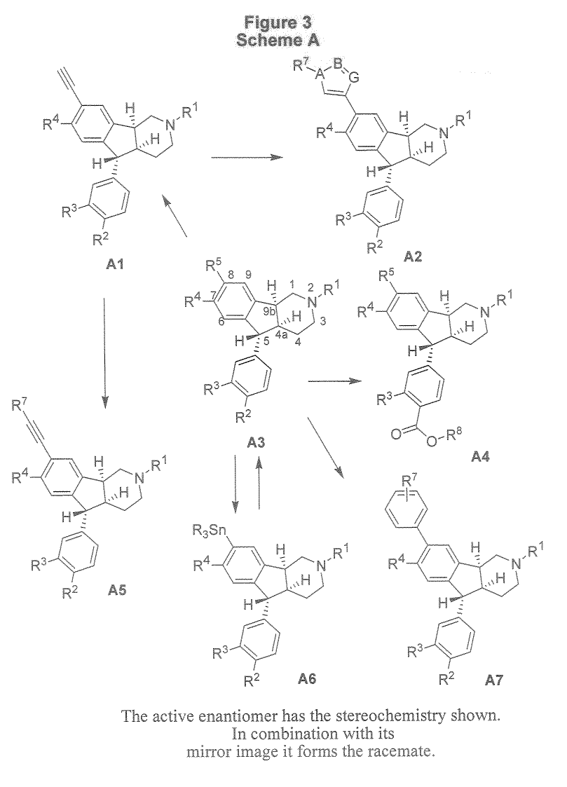

Designation of specific manufacturers, instruments, chromatographic material, chemical suppliers, etc., is for illustration only and does not preclude the use of other, similar items. General reagents and solvents were purchased and used without further purification. Hexamethylditln, tetrakis(triphenylphosphine)palladium(0), and chloramine-T (N-chloro-p-toluenesulfonamide sodium salt, hydrate) were purchased from Aldrich Chemical Company and sodium metabisulfite from Fisher Scientific Company. Carrier-free [¹²⁵I]NaI was purchased from NEN® Life Science Products, Inc. as a solution in 0.1 N NaOH. Water was distilled and subjected to reverse osmosis purification prior to use. NMR spectra were determined at 300 MHz on a Braker Avance instrument. Positions of multiplets are designated at the approximate center of the muitiplet. Not all peaks are given for every compound. Mass spectra for exact masses were determined at the University of Michigan Instrument Services by use of positive ion electrospray with formic acid added. TLC was carried out on silica gel 60 F254 plates (EM Separations Technology). Stirring was by means of a Teflon®-coated magnetic stirring bar. See FIG. 3 for structures.

### Example 1

### (4aS, 5R, 9bS)-5-(4-Carbomethoxyphenyl)-2-ethyl-8-ethynyl-2,3,4,4a,5,9b-hexahydro-7-methyl-1H-indeno[1,2-c]pyridine [A1a (A1 where R¹=Et, R²═COOMe, R³═H, R⁴=Me)]

The hydrochloride salt of (4aS, 5R, 9bS)-5-(4-Carbomethoxyphenyl)-2-ethyl-2,3,4,4a,5,9b-hexahydro-8-iodo-7-methyl-1H-indeno[1,2-c]pyridine [A3a.HCl, (A3 HCl where R⁵=Et, R²═COOMe, R³═H, R⁴=Me, R⁵═I)] (1.1 g) (Cook, C. E.; Jump. J. M.; Zhang, P.; Stephens, J. R.; Lee, Y.; Fail, P. A.; Anderson, S. A. J. Med. Chem., 1997, 40, 2111-2112; U.S. Pat. No. 5,319,084) was partitioned between aqueous sodium bicarbonate (50 mL, 5% by wt.) and methylene chloride (3×30 mL). The extract was washed with water (30 mL) and brine (30 mL) and dried over sodium sulfate. Filtration and solvent evaporation yielded the free base A3a (1.0 g, 98% yield): TLC R_{f} 0.21 on Whatman® LK C₁₈F. methanol-water (9:1, v/v), For visualization, the TLC plates were viewed under short ultraviolet light and then stained with iodine; ¹H NMR (CDCl₃, 300 MHz) δ 1.12 (t, 3, J=7.2 Hz), 2.33 (s, 3), 2.43 (q, 2, J=7.2 Hz), 3.92 (s, 1), 4.17 (d, 1, J=9.78 Hz), 6.78 (s, 1), 7.22 (d, 2, J=8.2 Hz), 7.71 (s, 1, 9-H), 8.04 (d, 2, J=8.2 Hz).

The 8-ethynyl analog A1a was prepared from A3a by use of a procedure similar to that described by Negishi, E.; Kotora, M.; Xu, C. J. Org. Chem., 1997, 62 (25), 8957-8960. Zinc bromide (1.0933 g, 4.86 mmol, anhydrous, Aldrich catalog no. 45,139-8, clear to opaque beads) was weighed under dry nitrogen, placed in an oven-dried three-neck round-bottom flask at room temperature under dry argon and dissolved in tetrahydrofuran (THF, 8.05 mL, Aldrich 99.9%, anhydrous, inhibitor free, catalog no. 186562). Ethynylmagnesium bromide (4.86 mmol, 9.71 mL of a 0.54 M solution in THF, Aldrich catalog no. 346152) was added at a fast dropwise rate at room temperature as the reaction mixture was stirred to obtain a milky suspension of ethynylzinc bromide. An aliquot (11.33 mL) of the well-stirred suspension was transferred with a gas-tight syringe to a three-neck round-bottom flask. Under dry argon, A3a (0.979 g, 2.06 mmol) dissolved in THF (15 mL) was added in one portion, followed by 5 mL of THF rinse, as the reaction mixture was stirred. After 5 min,

tetrakis(triphenylphosphIne)palladium(0) (118 mg, Aldrich catalog no. 02105PS, 99%, bright yellow) was added in one portion. When an aliquot work-up indicated significant starting material remained, more of the ethynylzinc bromide preparation (4.8 mL) was added and the reaction continued overnight. The next morning the remaining reaction mixture was poured into aqueous sodium bicarbonate (100 mL. 5% by wt.) and extracted with methylene chloride (3×50 mL). The extract was washed with water (50 mL) and brine (50 mL) and dried over sodium sulfate. Filtration and solvent evaporation yielded crude product (0.98 g). By TEC analysis no starting A3a was detectable, although more impurities (polar and non-polar) were observed than after 3 h. The crude product was chromatographed sequentially on two different columns. Whatman® LRP-2 (7 g, RP-18, 37-53 ix, catalog no. 4776-005) was equilibrated with methanol and packed in a 1 cm diameter flash chromatography column. Crude product was dissolved in methanol-methylene chloride (12 mL:3 mL) and placed on the column which was eluted with methanol. On the basis of TLC analysis, impure desired product (0.72 g) eluted in the 0-50 mL fraction. Silica gel 60 for column chromatography (15 g, 230-400 mesh, Merck KGaA) was equilibrated with methylene chloride and packed in a 2.5 cm diameter flash chromatography column. The impure product from the Whatman column was dissolved in a minimum of methylene chloride and placed on the column, which was eluted with a step-wise gradient of methylene chloride (100 mL) to ethyl acetate-methylene chloride (5:95, v/v, 100 mL) to ethyl acetate-methylene chloride (1:9, v/v, 100 mL) to ethyl acetate-methylene chloride (2:8, v/v, 100 mL) to ethyl acetate-methylene chloride-ethanol (2:8:0.25, v/v/v, 100 mL) to ethyl acetate-methylene chloride-ethanol (2:8:0.5, v/v/v, 100 mL) to ethyl acetate-methylene chloride-ethanol (2:8:1, v/v/v, 100 mL). On the basis of TLC analysis, four fractions eluted that contained the desired 8-ethynyl analog Ala: 123 mg in the 500 to 520 mL fraction; 173 mg in the 520 to 600 mL fraction; 54 mg in the 600 to 700 mL fraction; 48 mg in the 700 to 740 mL fraction (total of 398 mg, 22% yield), ¹H NMR analysis showed the intermediate fractions to be highly pure, with the remainder being suitable for further purification and synthesis: TLC R_{f} 0.5 on Whatman LK CigF, methanol-water (1:9, v/v); ¹H NMR (CDCl₃, 300 MHz) δ 12 (t, 3, J=7.2 Hz), 2.35 (s, 3), 2.42 (q, 2, J=7.2 Hz), 3.25 (s), 3.92 (s, 3), 4.21 (d, 1, J=9.9 Hz), 6.74 (s, 1), 7.23 (d, 1, J=8.2 Hz), 7.38 (s, 1), 8.00 (d, 2, J=8.2 Hz); MS (Positive Ion Electrospray with formic acid added) [M+H]⁺374.2131, C₂₅H₂₇NO₂ requires [M+H]⁺=374.2120.

### Example 2

### (4aS, 5R,9bS)-5-(4-Carbomethoxyphenyl)-2-ethyl-2,3,4,4a,5,9b-hexahydro-7-methyl-8-(1-(2-piperidin-1-yl)ethyl)-1H-1,2,3-triazole-4-yl)-1H-ideno[1,2-c]pyridine [A1a (A2 where R¹=Et, R²═COOMe, R³═H, R⁴=Me, ABG=N—N═N, R⁷═2-(N-piperidino)ethyl))].

1-(2-Azidoethyl)piperidine was obtained by a procedure similar to that described by Converso, A.; Burrow K.; Marzinzik, A.; Sharpless, B. K.; Finn. M. G. J. Org. Chem. 2002, 66 (12), 4386-4392. To sodium azide (2.00 g, 30.8 mmol, MW 65.01) dissolved in N,N-dimethylformamide (DMF, 30 mL) was added 1-(2-chloroethyl)piperidine hydrochloride (3.77 g, 20.4 mmol) and potassium hydroxide (1.38 g, 24.6 mmol). The stirred reaction mixture was refluxed for 2 h, cooled to room temperature, poured into water at 0-5° C. and extracted with ethyl ether (50 mL and 4×20 mL). The extract was washed with brine (50 mL) and dried over sodium sulfate. Filtration and solvent evaporation yielded crude product (2.77 g) that contained starting material. Treatment of the crude products dissolved in DMF (30 mL) with sodium azide (2.00 g) for 72 h at room temperature and work-up as described above yielded 1-2-(azidoethyl)piperidine of sufficient purity for use as an intermediate: ¹H NMR (CDCl₃, 300 Mz) δ 1.43 (m, 2), 1.59 (m, 4), 2.42 (apparent br t, 4, J=5 Hz), 2.55 (t, 3, J=6.3 Hz), 3.34 (t, 3, J=6.3 Hz).

The triazole analog Ala was prepared by use of a procedure similar to that described by Rostovtsev, V. V.; Green, L. G.; Fokin, V. V.; Sharpless, B. K. Angew. Chem. Int. Ed., 2002, 41 (14), 2596-2599. Ethynyl analog Ala (45 mg, 0.12 mmol) was dissolved in t-butanol containing 1-(2-azidoethyl)piperidine (15.4 mg, 0.12 mmol, 0.5 mL of a 4.3 mg/0.14 mL solution) and water (0.5 mL) added in 100 uL portions, resulting in an amber reaction mixture. An aqueous solution of copper(II) sulfate (8.0 μL of a 0.15 mmol/mL solution) was added followed by an aqueous solution of sodium L-ascorbate (12.04 μL of a 1 M solution). The reaction mixture was stirred under argon at room temperature for 20 h, diluted with methanol (1 mL), placed on a Sep-Pak® Plus C18 cartridge (Water's part no. WATO20515, pre-equilibrated sequentially with methanol, water and methanol) and eluted with methanol (6 mL). The filtrate was concentrated to 4 mL and placed on a RediSep™ C-18 reverse phase column (43 g, ISCO catalog no. 68-2203-030) equilibrated first with methanol and then with methanol-water (9:1, v/v). The column was eluted with methanol/water (9:1, v/v) at a flow rate of 30 mL/min as the effluent was monitored at 240 nm. Fractions were combined to obtain a front cut, a center cut and an end cut for the peak suspected to contain desired Ala. ¹H NMR analysis of the end cut indicated a mixture of desired product A2a and starting Ala.

The front and center cuts were highly pure by TLC and ¹H NMR analyses and were combined (35.9 mg, 56% yield): TLC R_{f} 0.42 on Whatman L K C₁₈F, methanol-water (9:1, v/v); ¹H NMR (CDCl₃, 300 Mz) δ 1.11 (t, 3, J=7.1 Hz), 1.455 (m, 2), 1.58 (m, 4), 2.36 (s, 3), 2.46 (m), 2.81 (t, 2, J=6.2 Hz), 3.92 (s, 3), 4.27 (d, 2, J=9.9 Hz), 4.52 (t, 2, J=6.2 Hz), 6.80 (s, 1, 6-H), 7.28 (d, 2, J=7.2 Hz), 7.77 (s, 1), 7.87 (s, 1), 8.01 (d, 2, J=8.05 Hz); MS (Positive Ion Electrospray with formic acid added) [M+H]⁺528.3330. C₃₂H₄₁N₅O₂ requires=528.3339.

### Example 3

### (4aS, 5R,9bS)-5-(4-Carbomethoxyphenyl)-2-ethyl-2,3,4,4a,5,9b-hexahydro-7-methyl-8-(2-phenylethynyl)-1H-indeno[1,2-c]pyridine [A5a (A5, where R¹=Et, R²═COOMe, R³, R⁴=Me, K⁷=phenyl)]

Under anhydrous conditions in an argon atmosphere, tetrakis(triphenylphosphine)palladium(0) (5.8 mg, 0.005 mmol), was added to a 2-neck flask fitted with a magnetic stirring bar, stopper, reflux condenser and inlet tube for dry argon. CuI (2.9 mg, 0.015 mmol), 0.5 mL of dry benzene and 75 μL of iodobenzene (137 mg, 0.67 mmol) were added in succession and the mixture stirred for 5 min, whereupon 1.3 mL of Et₃N was added. The ethynyl compound Ala (70.2 mg. 0.19 mmol) was dissolved in 1 mL of dry benzene and added to the reaction mixture with a 0.3 mL rinse of dry benzene. The mixture was stirred and heated at ca. 45° C. for 1 hr. Monitoring by TLC (Whatman® LK C₁₈F reverse phase plates developed with 90 (MeOH:Et₃N, 100:1): 10H₂O (v/v) showed the reaction to be complete. The mixture was added to 7.5 mL of saturated aqueous NH₄Cl and 22.5 mL of H₂O and extracted with CH₂Cl₂ (3×12 mL). Combined extracts were washed successively with H₂O (30 mL) and saturated aqueous NaCl (30 mL), dried over Na₂SO₄ and filtered. Residue from solvent evaporation (rotary evaporator) was dissolved in 5 mL of MeOH and passed through two reverse phase cartridges (Waters Sep-Pak® Plus C-18, pre-washed with MeOH, H₂O and MeOH) connected in series. Elution with methanol gave product in the first 15 mL (analyzed by TLC system above). This solution was concentrated to ca. 5 mL and chromatographed on a 43 g reverse phase (C18) column (Isco Redi-Sep™ C-18 RP column) by use of an automated chromatography system (Isco Flash Chromatography Companion, equilibrated with MeOH and then with MeOH:H₂O (9:1, v:v)). Elution beginning with MeOH:H₂O (9:1, v:v, solvent B) for 8 column volumes, followed by a linear gradient to 80% solvent A and 20% MeOH over 4.4 column volumes, with monitoring by UV absorbance at 240 nm, eluted the desired product in column volumes 7-10. A center cut of this fraction gave, after solvent evaporation, 38.6 mg of product (46% yield): ¹H NMR (CDCl₃, 300 Mz) δ 1.12 (t, 3, J=7 Hz), 1.64 (m, 2), 1.90 (m, 2), 2.20 (m, 1), 2.42 (m, 5), 2.52 (m, 1), 2.76 (m, 1), 2.94 (m, 1), 3.92 (s, 3), 4.23 (d, 1, J=10 Hz), 6.77 (s, 1), 7.25 (d, 2, J=8 Hz, partial overlap with CHCl₃ peak), 7.35 (m, 3), 7.42 (s, 1), 7.52 (m, 2), 8.01 (d, 2, J=8 Hz). MS (Positive Ion Electrospray with formic acid added) [M+H]⁺450.2427, C31H₃₁NO₂ requires=450.2433.

### Example 4

### (4aS, 5R,9bS)-5-(4-Carbomethoxyphenyl)-2-ethyl-2,3,4,4a,5,9b-hexahydro-7-methyl-8-{2-[4-(N-pyrrolidinonyl)phenyl]ethynyl}-1H-indeno[1,2-c]pyridine [A5b (A5, where R¹=Et, R²═COOMe, R³═H, R⁴=Me, R⁷=4-(N-pyrrolidinonyl)phenyl)].

Under anhydrous conditions in an argon atmosphere, tetrakis(triphenylphosphine)palladium(0) (5.1 mg, 0.0044 mmol), was added to a 2-neck flask fitted with a magnetic stirring bar, stopper, reflux condenser and inlet tube for dry argon. CuI (2.5 mg, 0.013 mmol) and 0.5 mL of dry benzene were added in succession, followed by 1-(4-iodophenyl)pyrrolidin-2-one [M. S. Manhas and S. J. Jeng. J. Org. Chem., 32:1246 (1967)](crystallized from ethanol and dried in vacuo, 175 mg, 0.61 mmol), and 0.5 mL of benzene and the mixture stirred and warmed gently until most of the iodo compound dissolved. The ethynyl compound Ala (61 mg, 0.16 mmol) was dissolved in 1 mL of dry benzene and added to the reaction mixture with a 0.3 mL rinse of dry benzene. The mixture was stirred for 5 min, whereupon 1.3 mL of Et₃N was added, and then heated at ca. 45° C. for 1 hr. A small aliquot was worked up (see below). Monitoring by TLC (Whatman® LK C₁₈F reverse phase plates developed with 90 (MeOH:Et₃N, 100:1): 10 H₂O (v/v) showed the reaction to be complete. The mixture was added to 10 mL of saturated aqueous NH₄Cl and 30 mL of H₂O and extracted with CH₂Cl₂ (3×10 mL). Combined extracts were washed successively with H₂O (10 mL) and saturated aqueous NaCl (10 mL), dried over Na₂SO₄ and filtered. Residue from solvent evaporation (rotary evaporator) was dried in vacuo overnight and then was dissolved in 5 mL of MeOH and passed through two reverse phase cartridges (Waters Sep-Pak® Plus C-18, pre-washed with MeOH. H₂O and MeOH) connected in series. Elution with methanol gave product in the first 15 mL (analyzed by TLC system above). This solution was concentrated to ca. 5 mL and chromatographed on a 43 g reverse phase (C18) column (Isco Redi-Sep™ C-18 RP column) by use of an automated chromatography system (Isco Flash Chromatography Companion, equilibrated with MeOH and then with MeOH:H₂O (9:1, v:v)). Elution with MeOH:H₂O (9:1, v:v), with monitoring by UV absorbance at 240 nm, gave the desired product (TLC analysis). Solvent evaporation and drying in vacuo yielded 50.9 mg of product (59% yield): ¹H NMR (CDCl₃, 300 Mz) δ 1.12 (t, 3, J=7 Hz), 1.64 (m, 2), 1.91 (m, 2), 2.18 (m, 3), 2.43 (m, 5), 2.53 (m, 1), 2.64 (t, 2, J=8 Hz), 2.76 (m, 1), 2.94 (m, 1), 3.34 (m, 1), 3.89 (m, 5), 4.23 (d, 1, J=10), 6.76 (s, 1), 7.25 (d, 2, J=8 Hz, partial overlap with CHCl₃ peak), 7.41 (s, 1), 7.51 (d, 2, J=9 Hz), 7.64 (d, 2, J=9 Hz), 8.01 (d, 2, J=8 Hz). MS (Positive Ion Electrospray with formic acid added) [M+H]⁺533.2797, C35H₃₆N₂O₃ requires=533.2804.

### Example 5

### (4aRS,5SR,9bRS)-5-(4-carbomethoxyphenyl)-2-ethyl-2,3,4,4a,5,9b-hexahydro-7-methyl-8-trimethylstannyl-1H-indeno[1,2-c]-pyridine [racemic A6a (A6, where R¹=Et, R²═COOMe, R³═H, R⁴═R=Me)] and <4aS,5R,9bS)-5-(4-carbomethoxyphenyl)-2-ethyl-2,3,4,4a,5,9b-hexahydro-7-methyl-8-trimethylstannyl-1H-indeno[1,2-c]pyridine [enantiomer A6a (A6, where R¹=Et, R²═COOMe, R³═R=Me)]

In a procedure similar to that described by Zhong U. Pravin K, Wyrick, C D, Seltzman H H, Kepler J A, Boja J W, Kuhar M J, Carroll F I. J Label Compd Radiopharm 1999; 42:281-286, to racemic A3a (35 mg, 0.074 mmol), dissolved in toluene (2 mL), under anhydrous conditions and an argon atmosphere was added hexamethyklitin (62 mg, 0.19 mmol.) and tetrakis(triphenylphosphine)palladium(0) (1 mg). After the reaction mixture was refluxed for 6 h, it was stirred over a weekend at room temperature. Purification by silica gel 60 column chromatography [10 g, 230-400 mesh, E. Merck, 2.2 cm diameter column, methanol-chloroform (5:95, v/v)] yielded racemic A6a (25 mg, 66% yield). ¹H NMR (CDCl₃) δ 0.34 (s, 9), 1.16 (t, 3, J=7.2 Hz), 2.35 (s, 3), 2.47 (q, 2, J=7.2 Hz), 3.94 (s, 3), 6.76 (s, 1, 6-H), 7.28 (d, 2, J=8.2 Hz), 7.33 (s, 1, 9-H), 8.02 (d, 2, J=8.2 Hz); MS m/z 513 (82), 511(64), 72 (100), C26H₃₅NO₂Sn requires a mass of 513. By HPLC analysis, on a Waters Radial Pak A column [C-18, 8×100 mm, 10 μm, UV detector at 240 nm, 1 mL/min methanol-triethylamine (100:1, v/v)], racemic A6a was 95% AUG pure, with starting racemlc A3a the major by-product: t_{R} 7.09 min for A6a and 6.22 min for A3a.

To remove residual A3a, the material from the silica gel 60 column was dissolved in methanol at 10 mg/mL, and aliquots of 0.2 mL were injected onto a 25×100 mm Prep Pak μBondapak™ (C-18, 125 Å, 10 μm) column fitted with a 25×10 mm Guard-Pak™ μBondapak™ (C-18, 125 Å, 10 μm) column and eluted with methanol-triethylamine (100:1, v/v; 7 mL/min); UV detector at 240 nm. The chromatography system consisted of a Thermo Separations (Spectra Physics) P100 pump. Rheodyne 7125 injector and a Thermo Separations (Spectra Physics) UV100 variable wave-length detector at 240 nm. Racemate A6 (17.8 mg, 47.3% yield) eluted at about 10 min. No A3a was detectable in this material by analytical HPLC at the usual HPLC settings, although a trace of a peak at t_{R} 6.22 min was observable when the A6 peak was run well off scale.

Similarly, optically pure enantiomer (4aS,5R,9bS)-5-(4-carbomethoxyphenyl)-2-ethyl-2,3,4,4a,5,9b-hexahydro-8-iodo-7-methyl-1H-indeno[1,2-c]pyridin , 22.8 mg, 0.048 mmol) was used to synthesize the enantiomer trimethylstannyl derivative A6a, [(4aS,5R,9bS)-5-(4-carbomethoxyphenyl)-2-ethyl-2,3,4,4a,5,9b-hexahydro-7-methyl-8-trimethylstannyl-1H-indeno[1,2-c]pyridine] with equivalent results (10.2 mg, 42% yield).

### Example 6

### (4aS,5R,9bS)-5-(4-carbomethoxyphenyl)-2-ethyl-2,3,4,4a,5,9b-hexahydro-8-[¹²⁵I]iodo-7-methyl-1H-ideno[1,2-c]pyridine (enantiomer [¹²⁵I]-A3a) and (4aSR,5RS,9bSR)-5-(4-carbomethoxyphenyl)-2-ethyl-8-iodo-7-methyl-2,3,4,4a,5,9b-hexahydro-1H-indeno[1,2-c]pyridine (racemic A3a).

All glassware used in reactions involving a few μg of material was silanized by adding dichlorodimethylsilane in toluene (5:95, v/v) to the glassware and after 10 min, rinsing once with methanol and twice with toluene before the glassware was oven-dried at 120° C.

By a procedure similar to that described by Zhong D. Pravin K, Wyrick, C D, Seltzman H H, Kepler J A, Boja J W, Kuhar M J, Carroll F I. J Label Compd Radiopharm 1999; 42:281-286, twenty millicuries of carrier-free [¹²⁵I]NaI of specific activity 17.4 Ci/mg (0.0092 μmol) in 60 μL of 0.1 N NaOH was vortexed in the shipment vial and transferred along with 40 μL of 0.1 N NaOH rinse to a 1-mL conical reaction vial. Enantiomer A6a (30 μg, 0.0585 (μmol) in 30 μL of methanol-acetic acid (95:5, v/v) was added followed by 70 μL of methanol-acetic acid (95:5, v/v). Chloramine-T hydrate (4 μL of a 28.2 mg/10 mL aqueous solution,

0.05 μmol) was added and the capped (Teflon® liner) reaction mixture was mixed on a vortex mixer for 2-3 min. Sodium metabisulfite [4 μL of a 38 mg/10 ml, aqueous solution (0.08 μmol)] was added and the reaction mixture mixed on a vortex mixer for another 2-3 min. Preparative HPLC of the radioactive compound was accomplished with a system consisting of a Beckman model 110A HPLC pump. Waters U6K universal injector and Thermo Separation (Spectra Physics) variable wavelength monitor at 240 nm. A standard curve was generated by injecting 5, 7.5, 10, 12.5 and 15 μL of a 0.5 mg/mL solution of A3a (racemic free base) in methanol in duplicate: Y=−1.78×10⁻⁴+2.78×10⁻¹⁰ (A) where Y=μmol of A3a and A=peak area; r²=0.994. As much as possible of the reaction mixture was injected onto a YMC RP-18 column (4.6×150 mm, 5 μm, UV detector at 240 nm, 1 mL/min, 10% H₂O-90% [methanol-triethylamine (100:1, v/v)] using an attenuation setting determined by generation of the standard curve to readily measure about 5 μg of A3a. From the standard curve it was determined that the peak at 6.67 min (collected from 6.38 to 7.97 min) contained 5.34 μg or 1.12×10⁻² μmol of desired [¹²⁵I]-A3a. (Under these chromatographic conditions, the trimethyltin derivative A6 had a retention time of ca. 11.4 min). The peak fraction was diluted to 2 mL with absolute ethanol. An aliquot of 20 μL was diluted to 10 mL with ethanol and 20-μL aliquots of the diluted sample were measured in triplicate on a gamma counter (75% efficiency). The specific activity was calculated to be 1496 mCi/μmol. The difference between this and the theoretical value for carrier free ¹²⁵I is believed to be due to contamination of the HPLC system with unlabeled material.

Simultaneously, racemic A6a was converted to racemic A3a by use of nonlabeled sodium iodide in order to check the reaction conditions and HPLC system prior to injecting the radioactive sample.

Radiochemical purity of >99% was determined by thin layer chromatography (TLC) by use of a Bioscan AR-2000 Radioactivity Imaging Scanner. [₁₂₅I]-A3a was co-spotted with nonlabeled A3a on a Whatman KC18F plate that was developed with methanol-triethylamine (100:1, v/v; R_{f}=0.46). The cospotted nonlabeled A3a was visualized under short wave UV light. A Packard Auto-gamma Scintillation Spectrometer 5135 was used to measure the radioactivity of the labeled product.

The remainder of the chromatographic fraction (1.96 mL) containing [¹²⁵I]-A3a was diluted to 10 mL with absolute ethanol to obtain a dilute solution of [¹²⁵I]-A3a for storage at −78° C.

### Example 7

### (4aS,5R,9bS)-5-(4-carbomethoxyphenyl)-2-ethyl-2,3,4,4a,5,9b-hexahydro-8-[¹²⁵I]iodo-7-methyl-1H-indeno[1,2-c]pyridine ([¹²⁵I]-A3b)

A 1-mL aliquot of the 10 mL solution of [¹²⁵I]-A3a was added to 0.25 mL of 1 N sodium hydroxide in a conical vial fitted with a triangular Teflon® coated stir bar. The reaction vial was capped with a Teflon®-lined cap and heated at 60° C.±5° C. for 18 h with stirring. After the mixture had cooled to room temperature, 0.25 mL of 1 N hydrochloric acid was added followed by 5-μL increments of 1 N hydrochloric acid (15 μL total added) until the pH of the reaction mixture was about 6 (determined by EM Science pH 0-14 strips). An aliquot of the reaction mixture was spotted over nonlabeled A3b on a Whatman KC18F TLC plate. After the plate was developed with 30% (v) H₂0-70% [methanol-triethylamine (100:1, v/v)], the cospotted nonlabeled A3b was visualized under short wave UV light (R_{f}=0.35). Analysis with a Bioscan AR-2000 Radioactivity Imaging Scanner indicated >99% radiochemical purity. The solution of [¹²⁵I]-A3b was calculated to contain 1.04 mCi/mL and was assumed to have the same specific activity as [¹²⁵I]-A3a.

### Example 8

### (4aRS, 5SR,9bRS-5-(4-Carboxylphenyl)-2-ethyl-2,3,4,4a,5,9b-hexahydro-8-iodo-7-methyl-1H-indeno-[1,2-c]pyridine n-Hexyl ester Hydrochloride [A4a (A4, where R¹=Et, R³═H, R⁴=Me, R⁵═I, R⁸=M-hexyl)]

(4aRS,5R,9bRS)-5-(4-Carboxylphenyl)-2-ethyl-2,3,4,4a,5,9b-hexahydro-8-iodo-7-methyl-1Hindeno-[1,2-c]pyridine hydrochloride (A3b HCl (A3 HQ, where R¹=Et, R²═COOH, R³═H, R⁴=Me, R⁵═I) was prepared by refluxing (4aRS, 5SR, 9bRS)-5-(4-Carboxylphenyl)-2-ethyl-2,3,4,4a,5,9b-hexahydro-8-iodo-7-methyl-1Hindeno-[1,2-c]pyridine methyl ester hydrochloride (A3a.HCl (A3.HCl, where R¹=Et, R₂═COOMe, R³═H, R⁴=Me, R⁵═I) (U.S. Pat. No. 5,319,084) with a 1:1 mixture of acetic acid and 6 N HCl overnight, concentrating the solution and allowing the product to crystallize. The carboxylic acid (75 mg, 0.15 mmol) was treated with SOCl₂ (2 mL) and 8 μL of N,N-dimethylformamide (DMF) and refluxed under anhydrous conditions (argon atmosphere) for 24 hr. The SOCl₂ was evaporated (rotary evaporator). CH₂Cl₂ (3 mL) was added and evaporated. Then to the residue was added a solution of 2.52 mg of 4-N,N-dimethylaminopyridine 2 mL of CH₂Cl₂, followed by 49 μL of pyridine and 105 μL of n-hexanol. The mixture was refluxed for 24 hr under argon and then 15 mL of CH₂Cl₂ and 25 mL of 5% (w/v) KHCO₃ added. The organic phase was separated and two more extractions with CH₂Cl₂ performed. Combined organic layers were washed with water (2×15 mL) and saturated NaCl (15 mL) and stirred over Na₂SO₄ for 1 hr. The solid was filtered off and the filtrate evaporated. The residue was evaporated twice with toluene (ca. 5-10 mL), followed by evaporation with 5-10 mL of CH₂Cl₂. The residue was dissolved in ca. 2 mL of ethyl acetate/CH₂Cl₂ (2:8, v:v), and placed on a column (1.4 cm diameter) of silica gel (36 g, 230-400 mesh,) that had been packed as a slurry with ethyl acetate CH₂Cl₂ (2:8, v:v) and eluted successively with ethyl acetate/CH₂Cl₂ (2:8, v:v, 16 mL), ethyl acetate/CH₂Cl₂/EtOH (20:80:5, v:v:v, 16 mL), and ethyl acetate/CH₂Cl₂/EtOH (20:80:10, v:v:v, 16 mL). Fractions were collected and analyzed by TLC (silica gel, ethyl acetate/CH₂Cl₂/EtOH (20:80:10, v:v:v). Like fractions were combined and the product obtained by evaporation. The residue was converted to the HO salt by taking it up in ca. 1 ml, of CH₂Cl₂, adding 0.2 ml, of a 1M solution of anhydrous HCl in Et₂O, mixing well and evaporating under a stream of nitrogen. Gentle warming and sonication dissolved the HCl salt in 0.5 mL of CH₂Cl₂. After keeping for overnight at room temperature, and a few hr in a freezer, ethyl acetate (0.25 mL) was added. After 5 days, the crystals of A4a.HCl were obtained by centrifugation and dried at 50° C. overnight, giving 28.3 mg (32% yield); ¹H NMR (CDCl₃, 300 Mz) δ 0.9 (t, 3, J=7 Hz), 1.14 (t, 3, J=7 Hz), 1.26-2.03 (m, 8), 2.27 (m, 1), 2.33 (s, 3), 2.48 (m, 3), 2.76 (m, 1), 2.95 (m, 1), 3.37 (m, 1), 4.16 (d, 1, J=10 Hz), 4.31 (t, 2, J=7 Hz), 6.75 (s, 1), 7.22 (d, 2, J=8 Hz), 7.71 (s, 1), 8 (d, 2, J=8 Hz); MS (Positive Ion Electrospray with formic acid added) [M+H]⁺546.1850, C₂₈H₃₆INO₂ requires [M+H]⁺=546.1869.

### Example 9

### (4aRS, 5SR, 9bRS)-5-(4-Carboxylphenyl)-2-ethyl-2,3,4,4a,5,9b-hexahydro-8-iodo-7-methyl-1H-indeno-[1,2-c]pyridine 3-hydroxypropyl ester Hydrochloride [A4b.HCl where R¹=Et, R³═H, R⁴=Me, R⁵═I, R⁸=3-hydroxypropyl)].

By a procedure similar to that for the compound in Example 8, but beginning with 450 mg of compound A3b.HCl, substituting 1,3-propanediol for hexanol, and increasing the reagents proportionately, the title compound was obtained (65% yield): ¹H NMR (CDCl₃, 300 Mz) δ 1.2 (t, 3, J=7.2 Hz), 1.66 (m, 1), 2.03 (m, 3), 2.33 (s, 3), 2.39 (m, 1), 2.58 (m, 4), 2.92 (m, 1), 3.07 (m, 1), 3.46 (m, 1), 3.78 (t, 2. J=6 Hz), 4.16 (d, 1, J=10.2 Hz), 4.5 (t, 2, J= 6 Hz), 6.77 (s, 1), 7.22 (d, 2, J=8.1 Hz); 7.73 (s, 1), 8 (d, 2, J=8.1 Hz); (Positive Ion Electrospray with formic acid added) [M+H]⁺520.1360 C₂₅H₃₀INO₃ requires [M+H]⁺=520.1349.

### Example 10

### (4aRS,5SR,9bRS)-5-(4-Carboxylphenyl)-2-ethyl-2,3,4,4a,5,9b-hexahydro-8-iodo-7-methyl-1H-indeno-[1,2-c]pyridine ((R)-2,2-Dimethyl-1,3-dioxolan-4-yl)methyl ester [A4c (A4, where R¹=Et, R³═H, R⁴=Me, R⁵═I, R⁸═((R)-2,2-Dimethyl-1,3-dioxolan-4-yl))]

By a procedure similar to that for the compound in Example 8, but beginning with 225 mg of compound A3b HCl, substituting S-(+)-2,2-dimethyl-1,3-dioxolane-4-methanol [5-(+)-Solketal®] for hexanol, increasing the reagents proportionately, but not converting to the HQ salt, the title compound was obtained (100 mg, 38% yield): ¹H NMR (CDCl₃, 300 Mz) δ 1.12 (t, 3, J=7 Hz), 1.39 (s, 3), 1.46 (s, 3), 1.65 (m, 2), 1.87 (m, 1), 1.95 (t, 1, J=11 Hz), 2.21 (m, 2), 2.33 (s, 1), 2.42 (q, 3, J=7 Hz), 2.52 (m, 2), 2.74 (m, 1), 2.9 (m, 1), 3.33 (m, 2), 3.85 (m, 1), 4.38 (d, 1, J=5.4 Hz), 4.39 (s, 1), 4.46 (m, 1), 6.77 (s, 1), 7.23 (d, 2, J=8 Hz), 7.71 (s, 1), 8.02 (d, 2, J=8 Hz), MS (Positive Ion Electrospray with formic acid added) [M+H⁺]576.1601 C₂₈H₃₄INO₄ requires [M+H]⁺=576.1611. (Note that the designation of the chiral center in the ester moiety changes from that of starting material due to change in precedence order of substituents.)

### Example 11

### (4aRS,5SR,9bRS)-5-(4-Carboxylphenyl)-2-ethyl-2,3,4,4a,5,9b-hexahydro-8-iodo-7-methyl-1H-indeno-[1,2-c]pyridine (R)-2,3-Dihydroxy propyl ester Hydrochloride [A4d.HCl(A4.HCl, where R¹=Et, R³═H, R⁴=Me, R⁵═I, R⁸═((R)-2,3-dihydroxypropyl))]

Ca. 70-75 mg of the compound from Example 10 was dissolved in 2 mL of dioxane followed by 0.2 mL of water and 1 mL of 1M HCl in Et₂O. The reaction mixture was stirred in a stoppered flask under argon for ca. 2 hr. Solvents were evaporated and the residue dried in vacuo at ambient temperature. CH₂Cl₂ was added (5 mL) and evaporated. The residue was dissolved in 2-3 mL of water and the solution lyophilized to give 65 mg (essentially quantitative yield) of the title compound: ¹H NMR (CDCl₃/CD₃OD (9:1, v:v), 300 Mz) δ 1.48 (m, 3), 1.85 (m, 1), 2.34 (s, 3), 2.58 (m, 2), 2.75 (m, 1), 2.97 (m, 1), 3.14 (m, 2), 3.67 (m, 4), 3.96 (m, 2), 4.17 (d, 1, J=10.7 Hz), 4.34 (d, 2, J=5.4 Hz), 6.76 (s, 1), 7.27 (d, 2, J=7.7 Hz), 7.8 (s, 1), 8.06 (d, 2, J=7.6 Hz), MS (Positive Ion Electrospray with formic acid added) [M+H]⁺536.1277, C₂₅H₃₀INO₄ requires [M+H]⁺536.1298.

### Example 12

### (4aRS,5SR,9bRS)-5-(4-Carboxylphenyl)-2-ethyl-2,3,4,4a,5,9b-hexahydro-8-iodo-7-methyl-1H-indeno-[1,2-c]pyridine ((S)-2,2-Dimethyl-1,3-dioxolan-4-yl)methyl ester [A4e (A4, where R¹=Et, R³═H, R⁴=Me, R⁵═I, R⁸═((R)-2,2-Dimethyl-1,3-dioxolan-4-yl))].

By a procedure similar to that for the compound in Example 10, but beginning with 150 mg of compound A3b.HCl, substituting 5-(+)-2,2-dimethyl-1,3-dioxolane-4-methanol [R-(−)-Solketal®] for its R-isomer, and changing the reagents proportionately, the title compound was obtained (109 mg, 63% yield): ¹H NMR (CDCl₃/CD₃OD (9:1, v:v), 300 Mz) δ 1.12 (m, 3), 1.39 (s, 3), 1.46 (s, 3), 1.62 (m, 1), 1.93 (m, 3), 2.22 (m, 1), 2.33 (s, 3), 2.42 (q, 2, J=7 Hz), 2.51 (m, 1), 2.7 (m, 1), 2.89 (m, 1), 3.33 (m, 1), 3.87 (m, 1), 4.15 (m, 2), 4.38 (d, 1, J=5.3 Hz), 4.39 (s, 1), 4.46 (m, 1), 6.77 (s, 1), 7.23 (d, 2. J=8.2 Hz), 7.71 (s, 1), 8.01 (d, 2, J=8.2 Hz), MS (Positive Ion Electrospray with formic acid added) [M+H]⁺576.1590, C₂₈H₃₄INO₄ requires [M+H]⁺576.1611. (Note that the designation of the chiral center in the ester moiety changes from that of starting material due to change in precedence order of substituents.)

### Example 13

### (4aRS,5SR,9bRS)-5-(4-Carboxyphenyl)-2-ethyl-2,3,4,4a,5,9b-hexahydro-8-iodo-7-methyl-1H-indeno-[1,2-c]pyridine(S)-2,3-Dihydroxypropyl ester Hydrochloride [A4f.HCl(A4HCl, where R¹=Et, R³═H, R⁴=Me, R⁵═I, R⁸═((S)-2,3-dihydroxypropyl))]

By a procedure like that described in Example 11, but starting with 62 mg (0.11 mmol) of compound A4e from Example 12, was obtained 51 mg (88% yield) of the title compound: ¹H NMR (CDCl₃/CD₃OD (9:1, v:v), 300 Mz) δ 1.48 (m, 3), 1.85 (m, 1), 2.34 (s, 3), 3.14 (m, 2), 3.96 (m, 3), 4.17 (d, 1, J=11 Hz), 4.39 (d, 2, J=5.4 Hz), 6.76 (s, 1), 7.27 (d, 2, J=7 Hz), 7.8 (s, 1), 8.06 (d, 2, J=7 Hz), MS (Positive Ion Electrospray with formic acid added) [M+H]⁺536.1290, C₂₅H₃₀INO₄ requires [M+H]⁺=536.1298.

Obviously, numerous modifications and variations of the present invention are possible in light of the above teachings. It is therefore to be understood that, within the scope of the appended claims, the invention may be practiced otherwise than as specifically described herein

1. A compound of structure 1, 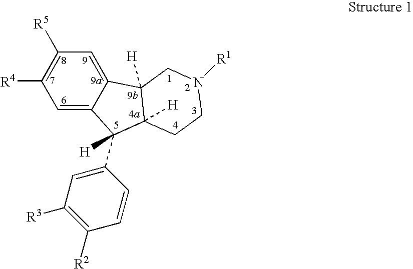
where R¹ is (optionally substituted) C₁₋₄ alkyl, C₃₋₆ cycloalkyl, C₂₋₄ alkynyl, C₂₋₄alkenyl;
R² is a carboxylic ester group in which the alcohol portion of the group has polar substituents or substitution; or
when R⁵ is not Cl, Br, or ¹²⁷I, R² is a carboxylic acid, carboxylic ester or a group that can be converted in vivo to a carboxylic acid;
R³ is H, halogen, (optionally substituted) C₁₋₄ alkyl, C₃₋₆ cycloalkyl, C₂₋₄ alkynyl, C₂₋₄alkenyl, or OR⁶, where R⁶ is H, C₁₋₄alkyl, perfluoroalkyl or CₙH_{1 to 2n+1}CO (which may be substituted with fluorine), where n=1 to 8;
R⁴ is C₁₋₄ alkyl, C₃₋₆ cycloalkyl, C₂₋₄ alkynyl, or C₂₋₄ alkenyl, any of which may be substituted with fluorine;
R⁵ is a group containing combinations of C, H, N, O, and S, characterized by one or more π-bonds, which group as it rotates around the C-8/R⁵ bond sweeps out a volume not greater than that of a cube of dimensions x=y=z=7 Angstroms and which group in addition may be substituted with a group R⁷ that can extend beyond this volume, where R⁷ is a combination of carbon, hydrogen, nitrogen, oxygen and sulfur atoms CₘHₒNₚO_{q}Sᵣ where m=0 to 12, o=0 to 25, p=0 to 5, q=0 to 5, and r=0 to 2; or
R⁵ is R¹¹R¹²R¹³Sn— where R¹¹R¹², and R¹³ are each, independently, C₁₋₆alkyl, C₂₋₆-alkenyl or C₂₋₆-alkynyl; or
R⁵ is ¹²⁵I, ¹²³I, ¹³¹I, ⁷⁶Br, ⁷⁷Br, ¹⁸F, ¹⁹F or ³H; or
when R² is not COOH, COO—CₙH₂ₙ₊₁ (where n is an integer from 1 to 4), CH₂OH, or CHO, R⁵ is Cl, Br, or ¹²⁷I
and salts thereof.

2. A compound of claim 1, where
R¹ is (optionally substituted) C₁₋₄ alkyl, C₃₋₆ cycloalkyl, C₂₋₄ alkynyl, C₂₋₄ alkenyl;
R² is a carboxylic acid ester wherein the alcohol portion of the ester contains ketal and/or hydroxyl-substituents or a group capable of being converted in vivo to hydroxyl; or
R² (when R⁵ is not F, Cl, Br, or ¹²⁷I) is a carboxyl or carboxyl ester group COO—CₙH₂ₙ₊₁ (where n is an integer from 0 to 4), CH₂OH, or CHO;
R³ is II, halogen, (optionally substituted) C₁₋₄ alkyl, C₃₋₆ cycloalkyl, C₂₋₄ alkynyl, C₂₋₄alkenyl, or OR⁶, where R⁶ is H, C₁₋₄ alkyl, perfluoroalkyl or CₙH_{1 to 2n+1}CO (which may be substituted with fluorine), where n=1 to 8;
R⁴ is C₁₋₄ alkyl, C₃₋₆ cycloalkyl, C₂₋₄ alkynyl, or C₂₋₄ alkenyl, any of which may be substituted with fluorine;
R⁵ is halogen (where R² is not a carboxylic acid, carboxylic ester or a group that can be converted in vivo to a carboxylic acid), or R⁵ is azido or cyano or a group containing C2=alkynyl, C₂₋₄ alkenyl, single ring aryl or single 5- or 6-membered ring heteroaryl or dihydroheteroaryl, wherein the said group may be substituted with a group R⁷, where R⁷ is a combination of carbon, hydrogen, nitrogen, oxygen and sulfur atoms CₘHₒNₚO_{q}Sᵣ where m=0 to 12, o=0 to 25, p=0 to 5, q=0 to 5, and r=0 to 2; or
R⁵ is R¹¹R¹²R¹³Sn— where R¹¹, R¹², and R¹³ are each, independently, C₁₋₆-alkyl, C₂₋₆-alkenyl or C₂₋₆-alkynyl; or
R⁵ is ¹²⁵I, ¹²³I, ¹³¹I, ⁷⁶Br, ⁷⁷Br, ¹⁸F, ¹⁹F or ³H;
and salts thereof.

3. A compound of claim 1, where
R¹ is (optionally substituted) C₁₋₄ alkyl, C₃₋₆ cycloalkyl, C₂₋₄ alkynyl, C₂₋₄ alkenyl;
R² is COOR⁸, wherein R⁸ is 2-hydroxypropyl, 3-hydroxypropyl, 2,3-dihydroxypropyl, or CH₂₋(2,2-dimethyl-1,3-dioxolan-4-yl), where R⁸ may be R, S or R,S if a chiral center is present;
or if R³ is not Cl, Br or ¹²⁷I, R⁸ is H or CₜHᵤ, where t=an integer from 1-18 and u=an Integer from 3-37;
R³ is H, halogen, (optionally substituted) C₁₋₄ alkyl, C₃₋₆ cycloalkyl, C₂₋₄ alkynyl, C₂₋₄alkenyl, or OR⁶, where R⁶ is H, Cl_{—}4 alkyl, perfluoroalkyl or CₙH_{1 to 2n+1}CO (which may be substituted with fluorine), where n=1 to 8;
R⁴ is C₁₋₄ alkyl, C₃₋₆ cycloalkyl, C₂₋₄ alkynyl, or C₂₋₄ alkenyl, any of which may be substituted with fluorine;
R⁵ is a group containing C₂₋₄ alkynyl C₂₋₄ alkenyl, single ring aryl or single 5- or 6-membered ring heteroaryl or dihydroheteroaryl, wherein the said group may be substituted with a group R⁷, where R⁷ is a combination of carbon, hydrogen, nitrogen, oxygen and sulfur atoms CₘHₒNₚO_{q}Sᵣ where m=0 to 12, o=0 to 25, p=0 to 5, q=0 to 5, and r=0 to 2; or
R⁵ is R¹¹R¹²R¹³Sn— where R¹¹, R¹², and R¹³ are each, independently, C₁₋₆alkyl, C₂₋₆-alkenyl or C₂₋₆-alkynyl; or
R⁵ is ¹²⁵I, ¹²³I, ⁷⁶Br, ⁷⁷Br, ¹⁸F, ¹⁹F or ³H; or
when R² is not COOH, COO—CₙH₂ₙ₊₁ (where n is an integer from 1 to 4), CH₂OH, or CHO), R⁵ is F, Cl, Br, or ¹²⁷I
and salts thereof.

4. A compound of claim 1, where
R¹ is (optionally substituted) C₁₋₄ alkyl, C₃₋₆ cycloalkyl, C₂₋₄ alkynyl, C₂₋₄ alkenyl;
R² is COOR⁸, wherein R⁸ is 2-hydroxypropyl, 3-hydroxypropyl, 2,3-dihydroxypropyl, or CH₂-(2,2-dimethyl-1,3-dioxolan-4-yl), where R⁸ may be R, S or R,S if a chiral center is present; or
when R⁵ is not Cl, Br, or ¹²⁷I, R²═COOR⁹, where R⁹ is H, methyl, ethyl or propyl;
R³ is H, halogen, (optionally substituted) C₁₋₄ alkyl, C₃₋₆ cycloalkyl, C₂₋₄ alkynyl, C₂₋₄alkenyl, or OR⁶, where R⁶ is H, C₁₋₄ alkyl, perfluoroalkyl or CₙH_{1 to 2n+1}CO (which may be substituted with fluorine), where n=1 to 8;
R⁴ is C₁₋₄ alkyl, C₃₋₆ cycloalkyl, C₂₋₄ alkynyl, C₂₋₄ alkenyl, which may be substituted with fluorine;
R⁵ is azido, cyano, ethynyl or a group containing C₂₋₄ alkynyl, C₂₋₄ alkenyl, single ring aryl or single 5- or 6-membered ring heteroaryl or dihydroheteroaryl, wherein the said group is substituted with a group R⁷, where R⁷ is a combination of carbon, hydrogen, nitrogen, oxygen and sulfur atoms CₘHₒNₚO_{q}Sᵣ where M=1 to 12, o=0 to 25, p=0 to 5, q=0 to 5, and r=0 to 2; or
R⁵ is ¹²⁵I, ¹²³I or ¹³¹I; or
when R² is not COOH, COO—CₙH₂ₙ₊₁ (where n is an integer from 1 to 4), CH₂OH, or CHO, R⁵ is F, Cl, Br, or ¹²⁷I;
and salts thereof.

5. A compound of claim 1, where
R¹ is methyl, ethyl, n-propyl, i-propyl, allyl or cyclopropyl;
R² is COOR⁸, wherein R⁸ is 2-hydroxypropyl, 3-hydroxypropyl, 2,3-dihydroxypropyl, or CH₂-(2,2-dimethyl-1,3-dioxolan-4-yl), where R⁸ may be R, S or R,S if a chiral center is present; or
when R⁵ is not Cl, Br, or ¹²⁷I, R² is COOR⁸, where R⁸ is H, methyl, ethyl or propyl;
R³ is H, halogen, CH₃, CF₃, CHO, CH₃CO, OH, OCH₃, or OCF₃;
R⁴ is CH₃, CF₃ or C₂H₅;
R⁵ is halogen, azido, cyano, ethynyl, propynyl, ethenyl, propenyl, triazol-4-yl, C₂₋₄alkenyl, single ring aryl or single 5- or 6-membered ring heteroaryl or dihydroheteroaryl, wherein the said group where chemically possible may be substituted with a group R⁷, where R⁷ is a combination of carbon, hydrogen, nitrogen, oxygen and sulfur atoms CₘHₒNₚO_{q}Sᵣ where m=o to 12, o=0 to 25, p=0 to 5, q=0 to 5, and r=0 to 2; or
when R² is not COOH, COO—CₙH₂ₙ₊₁ (where n is an integer from 1 to 4), CH₂OH, or CHO, R⁵ is F, Cl, Br, or ¹²⁷I; or
R⁵ is ¹²⁵I, ¹²³I or ¹³¹I,
and salts thereof.

6. A compound of claim 1, where
R¹ is ethyl;
R² is COOR⁸, wherein R⁸ is 2-hydroxypropyl, 3-hydroxypropyl, 2,3-dihydroxypropyl, or CH₂-(2,2-Dimethyl-1,3-dioxolan-4-yl), where R⁸ may be R, S or R,S if a chiral center is present;
R³ is H;
R⁴ is methyl;
R³ is halogen, ethynyl or (1-(2-(N-piperidino)ethyl))-1H-1,2,3-triazol-4-yl;
and salts thereof.

7. A compound of claim 1, where
R¹ is ethyl;
R² is COOR⁸, wherein R⁸ is H, methyl, ethyl, propyl, 2-hydroxypropyl, 3-hydroxypropyl, 2,3-dihydroxypropyl, or CH₂-(2,2-Dimethyl-1,3-dioxolan-4-yl), where R⁸ may be R, S or R,S if a chiral center is present:
R³ is H;
R⁴ is methyl;
R⁵ is ¹²³I, ¹²⁵I, ¹²⁷I, ¹³¹I, Me₃Sn, ethynyl or (1-(2-(N-piperidino)ethyl))-1H-1,2,3-triazol-4-yl;
and salts thereof.

8. A compound of claim 1, where
R¹ is ethyl;
R² is COOR⁸, wherein R⁸ is 2-hydroxypropyl, 3-hydroxypropyl, 2,3-dihydroxypropyl, or CH₂-(2,2-Dimethyl-1,3-dioxolan-4-yl), where R⁸ may be R, S or R,S if a chiral center is present; or
if R⁵ is not ¹²⁷I, R² is COOR⁸ where R⁸ is H, methyl, ethyl or propyl;
R³ is H;
R⁴ is methyl;
R⁵ is trimethylstannyl, ¹²³I, ¹²⁵I, ¹²⁷I, ¹³¹I, ethynyl or (1-(2-(N-piperidino)ethyl))-1H-1,2,3-triazol-4-yl;
and salts thereof.

9. The use of a compound of claim 1 for control of fertility in mammals by administration to said mammal of an effective dose of the compound.

10. Claim 9 where said mammal is a human male;

11. Claim 9 where said mammal is a feral or wild animal;

12. Claim 11 where said mammal is a mouse, rat, coyote, dingo, burro, deer, groundhog, coyote or horse.

13. A method for killing motile spermatozoa and/or rendering them immotile, comprising:
contacting a spermatozoa containing composition with a spermicidal composition comprising a compound of claim 1.

14. A spermicidal composition comprising an effective spermicidal dose of a compound of claim 1 and a physiologically acceptable carrier.

15. A spermicidally treated contraceptive device comprising:
an effective spermicidal amount of a compound of claim 1
and a carrier; and
a contraceptive barrier device.

16. A contraceptive method, comprising:
orally administering to a subject, a composition comprising an effective spermicidal amount of a first compound of claim 1:
in a pharmacologically acceptable carrier; and
concurrent use by said subject or the partner of said subject of a spermicidally treated contraceptive device comprising:
an effective spermicidal amount of a second compound and a carrier; and
a contraceptive barrier device;
wherein said first compound of formula I(a) and said second compound may be the same or different.

17. The use of a compound of claim 1, wherein R⁵ is R¹¹R¹²R¹³Sn— where R¹¹, R¹², and R¹³ are each, independently, C₁₋₆-alkyl, C₂₋₆-alkenyl or C₂₋₆-alkynyl, and R¹, R², R³ and R⁴ are as defined in claim 1 for the preparation of compounds of structure 1, wherein R⁵ is a radioactive atom and R¹, R², R³ and R⁴ are as defined in claim 1.

18. The use of a compound of claim 1, wherein R¹ is Et, R² is —COOMe or —COOH. R⁵ is H, R⁴ is Me and R⁵ is Me₃Sn—, for the preparation of a compound of structure 1 wherein R¹ is Et, R² is —COOMe or —COOH, R³ is H, R⁴ is Me and R⁵ is ¹²³I, ¹²⁵I, or ¹³¹I.

19. The use of a compound of claim 1, wherein R¹, R², R³, R⁴ and R⁵ are as defined in claim 1 and the compound is rendered radioactive by substitution with a radioactive atom, for identifying, locating or quantitating sites and molecules which bind antispermatogenic or spermicidal compounds.

20. The use of a compound of claim 1, wherein R¹, R², R³, R⁴ and R⁵ are as defined in claim 1 and the compound is rendered radioactive by substitution with a radioactive atom, for assays to Identify structure-activity relationships among possible antispermatogenic or spermicidal compounds and to aid in the identification and development of new antispermatogenic or spermicidal compounds.

21. Claim 19, where R¹ is Et, R² is —COOMe or —COOH, R³ is H, R⁴ is Me and R⁵ is ¹²³I, ¹²⁵I, or ¹³¹I.

22. Claim 20, where R¹ is Et, R² is COOMe or —COOH, R³ is H, R⁴ is Me and R⁵ is ¹²⁵I.

23. Claim 19, where the sites are in the testis or subtractions thereof.

24. Claim 21, where the sites are in the testis or subfractions thereof.

25. Claim 22, where the sites are in the testis or subfractions thereof.

26. An assay using a compound of claim 1, wherein R¹, R², R³, R⁴, and R⁵ are as defined in claim 1 and the compound is rendered radioactive by substitution with a radioactive atom, for determining the relative affinity of compounds for a receptor.

27. Claim 26, where the receptor is located in the testis.

28. Claim 26, where the receptor is in the membrane fraction of testicular homogenates or specific cellular components of the testis.

29. Claim 26. where R¹ is Et, R² is —COOH, R³ is H, R⁴ is Me and R⁵ is ¹²⁵I.

30. Claim 29, where the receptor is located in the testis.

31. Claim 29, where the receptor is in the membrane fraction of testicular homogenates or specific cellular components of the testis.
